(19) United States
(12) Patent Application Publication
(10) Pub. No.: US 2007/0207060 A1
(43) Pub. Date: Sep. 6, 2007

(54) Testing method of analytic chip of multiple reactors, the analytic chip, and the testing device

(75) Inventors: Fanglin Zou, Chengdu City (CN); Chunsheng Chen, Chengdu City (CN); Jianxia Wang, Meishan City (CN)

Correspondence Address: BIRCH STEWART KOLASCH & BIRCH, PO BOX 747, FALLS CHURCH, VA, 22040-0747, US

(73) Assignee: Chengdu Kuachang Medical Industrial Limited, Chengdu (CN)

(21) Appl. No.: 11/655,848

(22) Filed: Jan. 22, 2007

Related U.S. Application Data
(63) Continuation of application No. PCT/CN05/00412, filed on Mar. 29, 2005.

(30) Foreign Application Priority Data
Jul. 21, 2004 (CN) PCT/CN04/00839
Feb. 6, 2005 (CN) 200510020350.5

(51) Int. Cl. G01N 33/00

(52) U.S. Cl. 422/68.1

(57) ABSTRACT

This invention involves the testing device of analytic chip with multiple reactors, which includes a). one-way-cleaning system for one-way-cleaning of remnant sample in said reactor; or/and b).fluidity-decreasing system for fluidity-decreasing of remnant sample; or/and c). sample spotting system for sample subjecting by spotting. With the said testing device, analytic chip testing can be easily conducted with high integration, high efficiency and safety.

### CROSS-REFERENCE TO RELATED APPLICATIONS

This application is a Continuation of co-pending Application No. PCT/CN2005/000412 filed on Mar. 29, 2005, and for which priority is claimed under 35 U.S.C. §120; and claims priority under 35 USC 119 to Chinese Patent Application No. 200510020350.5 filed on Feb. 6, 2005; PCT/CN2004/000839 filed on Jul. 21, 2004, respectively, the entire contents of which are hereby incorporated by reference.

### BACKGROUND OF THE INVENTION

1. Field of the Invention

This invention concerns a testing device for analysis of analytic chip of multiple reactors.

2. Description of Background Art

In this invention, said analytic chip, or chip, refers to a testing material for qualitative and/or quantitative analysis. The chip includes chips with different forms, such as microchannels chip and microarray chip. The core of a chip is its reactor, while the core of reactor is the probe immobilized. The chip includes chips with different probe, such as biochip and non-biochip. At present, the chip used more commonly is biochip, and the biochip used more commonly is peptide chip and gene chip. The chip has extensive applications, including the fields of determination of gene expression, gene screening, medicine screening, diagnosis and treatment for diseases, control and administration for environment, identification in judicial affairs, etc.

In this invention, chip is defined as mono-reactor biochip (n=1) or chip of multiple reactors (n≧2), according to the number of reactors (n) in the chip. In testing by chip of multiple reactors, sample subjecting on the reactor and cleaning of the reactor with remnant sample are the most influential factors to the test efficiency and automation.

The testing device with sample subjecting system or/and reactor cleaning system is an important device for testing with chip of multiple reactors. Some of the biochip testing devices are made public, but only a few of the testing devices can be used for chip of multiple reactors.

Though reactor-cleaning by spurting has long been adopted in the testing of a chip with single reactor, some different technical designs have to be developed to meet the need of the automated cleaning in the chip of multiple reactors. For instance, the reactors on the chip are designed to have the same size and distance as the reaction wells on the micro-well plate. At the same time, ELISA testing device will help its automatic testing. However, since the micro-well plate washer is of two-way cleaning, consisting of sucking away the reaction residual and washing by injection, it is time-consuming and ineffective in cleaning.

The present sample-subjecting device is the device based on injection, such as pipette, sampling pump etc, which firstly inputs sample into a container (e.g. the inlet of injector or the tube of sampling pump) and then push it out of the container with mechanic force. This kind of devices is usually of technical complexity and hence takes long time to load sample.

Therefore, to find a new testing method with a higher integration, a higher efficiency and a higher sensibility, and to produce the chip and the testing device responsible for the testing method, are the urgent problems in the development of chip of multiple reactors.

In testing by chip of multiple reactors, the present sample subjecting is subjecting sample by injection, which means first mechanically sucking the liquid sample and then injecting it into reactors, e.g. the sample subjecting through injector, pipette or pump. The sample subjecting by injection consists of sucking and discharging the sample, thus is complicated and have many limits in application to chip of multiple reactors especially chip with high density of reactors (e.g. limit of space).

In testing by chip of multiple reactors, the present reactor cleaning, especially the present cleaning of reactor with reaction residual is of a two-way procedure, which comprises: a). removing the reaction residual from the reactor, b). subjecting the washing solution into the reactor first, and then removing the washing solution c).repeating the subjecting and removing of the washing solution for several times. This reactor cleaning is similar to the washing of ELISA plate. Since delicate instrument is required for the two-way cleaning, this method has many limits in application to chip of multiple reactors especially chip with high density of reactors (e.g. limit of space). In addition, with a small momentum of the washing solution, the washing effect is yet to be improved.

The chip of multiple reactors is an important material means for the said testing. The partition structure, which separates reactors, is the one of the most important structures of the chip of multiple reactors. In fact, the initial chip now in wide use is of no partition structure, usually a chip with single open non-flow reactor, such as the chip with microscope slide as substrate while with no other newly added structures, which is activated and spotted with probe. The advantage of this chip with single open non-flow reactor lies in its simple structure, easy sampling and scanning, with part of the operations carried out in the flowing media driven by the external force (e.g. washing by spraying). The disadvantage is that only one reactor can be used. When not many kinds of substance of interests (e.g. less than 100 kinds) are included in the sample, the chip of multiple reactors especially in high density should be developed so as to reduce chip cost and increase testing efficiency. The partition structure of the chip of multiple reactors is of the following properties: A. it can effectively restrict the flowing of liquid media so as to avoid cross-contamination or reaction. B. it should be as low as possible, which serves as an important approach to better washing reactors and decreasing the technical requirement for the scanning device. C. it will not react with media nor be contaminated. At present, the partition structure of the chip of multiple reactors is based on one of more of the following mechanisms: partition by height difference, partition by different plane and partition by using hydrophobic and superhydrophobic materials (PCT application: PCT/CN03/00055 and PCT/CN2004/000 169). However, these partition structures turn out to be defective in controlling the flowing of organic media and cross-contamination, when the height of partition structure is minimized.

### SUMMARY AND OBJECTS OF THE INVENTION

This invention is intended to achieve the high integration and high efficiency of the analytic chip testing at the premise of convenience and safety, wherein “high integration” means that a same process can be simultaneously carried out in several reactors especially the reactors with high density (e.g. the multiple reactors with the density over 1 reactor/cm²); “high efficiency” means short testing time or/and low testing cost or/and high sensitivity or/and frequent repetition; “safety” means that cross-contamination among reactors are controllable.

This invention is to provide a highly integrated and efficient testing device of chip of multiple reactors at the premise of easy operation and safety. This invention is based on an unexpected result produced in our study on cross-contamination among multiple reactors of an analytic chip.

In general view, the remnant sample in one reactor should be completely kept from entering another reactor so as to avoid the cross-contamination in the reactor cleaning. Though in analytic chip with single open reactor, fluid spurting is used to directly wash away the reaction residual, no similar application is adopted in case of analytic chip of multiple reactors. However, as shown in the implementation of this invention, contrary to the general view, even though without special partition structure to prevent reaction residual in one reactor from entering adjacent one, cross-contamination will not necessarily develop in the reactor-cleaning once the testing device with the reactor-cleaning system is employed to have a direct one-way spurting on the multiple reactors of said chip.

In addition, if a sample subjecting better controllable in size and position of sample (for example, the sample subjecting by sample spotting in the implementation of this invention), and/or a specific treatment of remnant sample (for example, the fluidity-decreasing procedure in the implementation of this invention) can be applied, the cross-contamination will be more controllable. And if the reactor-cleaning, sample subjecting by spotting and fluidity-decreasing procedure are combined in any way, cross-contamination can be further reduced.

So, this invention provides a testing method of analytic chip of multiple reactors, comprising at least one of the following steps or any combination of the following steps: a). sample subjecting by spotting; b). fluidity decreasing of remnant sample in said reactor; c). one-way cleaning of said reactor with remnant sample. In a testing method according to the testing method of this invention, said one-way cleaning comprises subjecting Q individual fluids to N reactors respectively, by which said remnant samples are washed away from said reactors, wherein: a). Q≧N≧2; and b). said Q fluids are subjected respectively to said N reactors at almost the same time.

This invention provides also a testing device of analytic chip of multiple reactors, comprising: a). one-way-cleaning system for one-way-cleaning of remnant sample in said reactor, wherein said one-way cleaning comprises subjecting Q individual fluids to N reactors respectively, by which said remnant samples are washed away from said reactors, wherein Q≧N≧2; or/and b). fluidity-decreasing system for fluidity-decreasing of remnant sample; or/and c). sample spotting system for sample subjecting by spotting.

As shown in the implementation examples of this invention, the said testing device of analytic chip is intended to achieve the following goals:

1). With the sample spotting system of this invention, a highly integrated, efficient and safe sampling can be conducted, wherein:

a). With the said sample spotting system, samples are loaded simultaneously to several reactors (e.g. 16-48 reactors), especially the reactors of high density (e.g. a chip with a reactor density over 1 reactor/cm²). Thus it is of high integration.

b). With the said sample spotting system, samples with definite volume, area and momentum can be subjected to multiple reactors by the said spotting, which will help control cross-contamination, increase sensitivity. Thus it is of a high efficiency.

c). With the said sample spotting system, the area and volume of samples in the reactors can be minimized so that cross-contamination can be effectively controlled. Thus it is of high safety.

2). With fluidity-decreasing system of this invention, a highly integrated, efficient and safe testing can be conducted, wherein:

a). With the said fluidity-decreasing system, the fluidity of various liquid media on several reactors (e.g. 16-48 reactors), especially the reactors of high density (e.g. a chip with a reactor density over 1 reactor/cm²) can be handled. Thus it is of high integration.

b). With the said fluidity-decreasing system, a short time is needed to control the fluidity of media. Thus it is of high efficiency.

c). With the said fluidity-decreasing system, the fluidity of the remnant samples can be reduced and the cross-contamination is under control. Thus it is of high safety.

3). With the one-way cleaning system of this invention, a highly integrated, efficient and safe testing can be conducted, wherein:

a). With the said one-way cleaning system, several reactors (e.g. 16-48 reactors), especially the reactors with high density (e.g. multiple reactors with a density over 1 reactor/cm²) can be cleaned at the same time. Thus it is of high integration.

b). With the said one-way cleaning system, the reactor cleaning can be completed within a short time (e.g. 1-10 seconds) and the washing effect is enhanced with the increase of linear speed of washing fluid. Thus it is of high efficiency.

c). With the said one-way cleaning system, the cross-contamination can be controlled. Thus it is of high safety.

4). When the said sample spotting system, fluidity-decreasing system and one-way cleaning system are combined in any way (spotting system with fluidity-decreasing system; sample spotting system with one-way cleaning system; fluidity-decreasing system with one-way cleaning system or sample spotting system with fluidity-decreasing system and one-way system etc.), they can achieve an even better result (e.g. an even higher efficiency or/and higher safety) without losing their own integration, efficiency and safety.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 shows an example of the chip of multiple reactors, which is composed of the substrate (1), the probe spots (2) immobilized thereon, and the reactor partition structure (3) linked thereon, wherein the reactor (4) containing the probe region (5) and the non-probe region (6).

FIG. 2(A). shows an example of the nozzle (7) with outlet (8) located on a convex body; FIG. 2(B). shows an example of the depressed nozzle (7) with outlet (8) located at a concave; FIG. 2(C).shows an example of the plane nozzle (7) with outlet (8) on the plane.

FIG. 3 shows the relationship among different systems of the testing device containing the reactor cleaning system adopted in application of this invention. The systems are: sample preparing system (9), sample loading system (10), sample reaction system (11), fluidity-decreasing system (12), reactor-cleaning system (13), signal reading system (14) and signal analyzing system (15).

FIG. 4 shows the working principle of the testing device with the reactor-cleaning system. Said testing device comprises the following systems: separate washing chamber (16), temperature/moisture controller (17), pressure pump (18), storing bottle for washing solution (19), pipeline (20), nozzle (21), outlet path (22) and controlling system (23).

FIG. 5 shows the sample spotting system based on one of the implementations of this invention. FIG. 5a is the fixturn of spotting tips (24), FIG. 5b shows different kinds of the spotting tips (26) that can be inlaid, wherein the fixturn of multiple spotting tips contains stem (25), and the spotting tip contains mortise (27). The end of spotting tip can be of various shapes, for instance, some have a slop (28), some have seam and sulcus, other are in shape of cast, hemisphere, tube, conus or cube. Sometimes it can be a combination of these shapes.

### DETAILED DESCRIPTION OF THE PREFERRED EMBODIMENTS

a. Terms

In describing the present invention, the following terms will be employed, and are defined as indicated below.

The term “analytic chip”, or “chip” in abbreviation, refers to a material that contains reactor for qualitative and/or quantitative analysis, on which probes, which can have specific reaction with substance of interest in sample with results identifiable in the address-traceable pattern, are immobilized. The chip in this invention includes the following examples: biochip, non-biochip, microarray, etc. In this invention, chip can be defined as chip with single reactor (containing only one reactor) or chip of multiple reactors (containing several reactors), according to the number of reactors in the chip.

In this invention, the chip of multiple reactors is composed at least of substrate, probe spots immobilized thereon, and reactor partition structure. The term “probe” refers to the substance immobilized on the substrate for capturing the substance of interest, such as DNA, polypeptide, protein, cell or tissue etc; the term “substrate” refers to solid support for immobilizing the probes; and the term “reactor partition structure”, or “partition structure” in abbreviation, refers to the structure used to separate a reactor from all other reactors, such as separating coat, separating zone or separating plate etc. In this invention, the said partition structure on the subject chips is to separate reactors mainly in the process of sampling and reaction, while in the cleaning of the reactor with remnant sample, it may or may not present this function.

In this invention, the term “analytic chip reactor”, or “reactor” in abbreviation, refers to the place and all the structures connected with the place, where probes are immobilized and made to react with substance of interest. An example of the reactor is the reactor including substrate, probes immobilized thereon, partition structure and other relevant structures connected (e.g. flowing path, inlet structure, outlet structure, immobilized marker etc.)

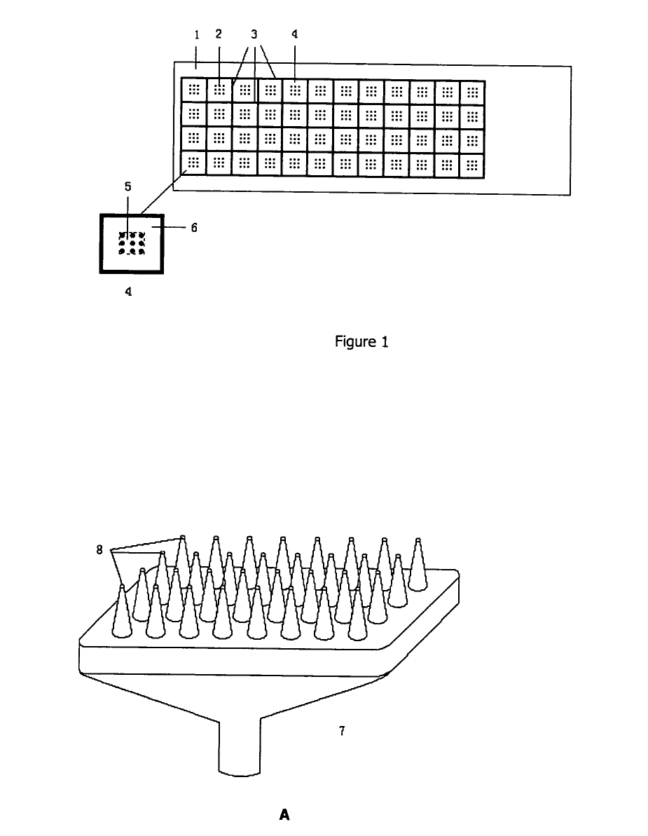

In this invention, the reactor comprises probe region and non-probe region. The term “probe region” refers to the region on the substrate where the probe spots are immobilized, whose boundery line is the line linking the probe spots closest to the partition structure, such as the region (5) in FIG. 1; and the term “non-probe region” refers to the region on the substrate in the reactor, between said probe region and the partition structure, such as region (6) in FIG. 1.

In this invention, the chip of multiple reactors can contain structure other than the probe spots, the substrate and the partition structure, such as the protective structure. The term “protective structure” refers to the structure linked to the chip, which can close part or all structures of the reactors in stage before the sample subjecting, (e.g. in transportation, storage etc.) which can be partially or wholly removed before the sample subjecting. The protective structure is designed to protect reactors on the chip from contamination when they are not in use. In the testing, the protective structure will be removed irreversibly either as a whole or in part so that the sampling is performed in the reactor. (Ref. PCT/CN2004/000169).

The term “analytical chip testing”, or “chip testing” in abbreviation, refers to testing by using devices including analytical chip, such as screening for blood transfusion by biochip with antigen/antibody probe.

In this invention, the reactor is classed according to its status in different stage of the analytic chip testing. For instance: a reactor is classed as protected reactor or non-protected reactor according to whether the reactor is protected by the protective structure or not in the transportation and/or storage of chip; a reactor is classed as either open reactor or closed reactor according to whether the probe region is exposed or not in the sample subjecting process; a reactor is classed as either flow reactor or non-flow reactor depending on whether the sample added is flowing as orientated or not on the probe region in the sampling process; a reactors is classed as either exposed reactor or unexposed reactor according to whether the remnant sample above the probe region is exposed or not in the reactor-cleaning process. A chip reactor may have different reaction status in same process and in different process, so may be classified as different reactor. For instance, a reactor can be a protected reactor when it contains protective structure in the course of transportation and/or the storage; an open reactor when probe region becomes open in the sampling process; an open flow reactor when probe region becomes open in the sampling process and the samples added flow in the set direction; an exposed reactor when probe region is open while washing reactor of remnant sample. As indicated in above examples, since reactor is classed in light of a certain status in a certain test process, a reactor thus may have different components in different processes. For instance, a protected reactor, consisting of a protective structure and a reversibly-closed structure in the storage, becomes a reversibly closed reactor when the protective structure has be removed in the sampling process, and becomes an exposed reactor when the reversibly-closed structure has been moved away in the reactor cleaning.

The term “plane reactor” refers to the reactor with the plane substrate; and the term “plane substrate” refers to substrate, whose surface for immobilizing the probes is or approximately horizontal in the reactor-cleaning process, such as activated glass slide and metal plate etc.

In this invention, chips are defined differently according to the reactors contained. For instance, the chips with open reactors, closed reactors, flow reactors or non-flow reactors are defined as open chip, close chip, flow chip or non-flow chip respectively.

The term “the contact angle” refers to the angle between the tangent line of gas-liquid interface and the tangent line of solid-liquid interface, when a liquid drop on a solid surface has reached thermodynamic balance.

The term “hydrophobic-lipophobic structure” refers to the structure, which is resistant from both water and oil.

The term “hydrophobic-lipophobic organic material” refers to the material containing hydrophobic and lipophobic organism, such as the hydrophobic-lipophobic macromolecular materials used in the implementation of this invention. The term “hydrophobic-lipophobic nano-material” refers to the material containing hydrophobic-lipophobic nanostructure, including the material containing hydrophobic-lipophobic nanoparticles, such as the hydrophobic-lipophobic material with nanoparticles used in the implementation of this invention.

The term “well” refers to the reaction region on the substrate immobilized with probe and its surrounding partition structure. The term “multiple wells” means more than one well.

The term “sample” refers to a solution probably comprising the substance of interest for testing, such as human serum, diluted human serum, marked human serum etc; the term “substance of interest” means what arouses interest in the testing, such as test target, test intermedium etc; the term “remnant sample” means the sample containing un-reacted substances after the reaction between the probe and the substance of interest. The term “cleaning of remnant sample” means cleaning the reactor after removing the remnant samples, such as all the remnant samples in the open reactor or the rest of remnant samples in the reversibly closed reactor (most of the remnant samples have already been removed before this process).

The term “fluidity-decreasing” means decreasing of the fluidity; the term “fluidity-decreasing additive” means the inert matter used to decrease the fluidity. The fluidity-decreasing additive includes soluble viscose agent, which may increase viscosity such as sugar, and insoluble additive that can adsorb liquid, such as resin, chromatographic gel, diatomaceous earth etc.

The term “sampling” or “sample subjecting” means subjecting the sample probably containing the substance of interest into the chip reactor.

The term “sample subjecting by injection” refers to injecting samples into a container (e.g. the inlet of injector or the tube of sampling pump) by certain mechanic devices such as injector or pump and then push them out of the container to said chip reactor with mechanic force. At present, sampling in the testing of chip of multiple reactors is largely conducted through injection, which concerns the size of sample only instead of the controlling of the position, size and momentum.

The term “sample subjecting by spotting” refers to subjecting the sample into the reactor by spotting. In this invention, the spotting is similar to the spotting adopted in chip preparation, which helps to subject probe solution to the substrate. The sample subjecting by spotting is different from the sample subjecting by injection, which is to inject the sample to the reactors by mechanic force. The sample spotting in this invention includes non-contact spotting (e.g. spotting by spurting) and contact spotting (e.g. spotting by making the spotting tip have a direct contact with the probe region and its adjacent area).

The term “sample subjecting by non-contact spotting” means a subjecting sample in which the spotting tip will not touch the probe region when, such as the inkjet spotting. The term “sample subjecting by contact spotting” means a subjecting sample in which the spotting tip will have a contact with the probe region when spotting. An example of the sample subjecting by contact spotting is that the liquid sample is transferred to the spotting tip without any mechanic help such as pump, pipette etc. (e.g. the transferring based on capillary phenomenon), and then the liquid sample is transferred to a specific position of the probe region in definite size (e.g. 2-3 times larger than the probe region) and at any momentum (e.g. the linear speed over 1-100cm/second).

The term “reactor cleaning” refers to removing the unnecessary substance (e.g. remnant sample, remnant marker, etc.) from the reactor and washing the reactor. The term “cleaning the reactor with remnant sample” refers to the reactor cleaning of the reactor that contains the remnant samples.

The term “two-way cleaning” refers to a reactor cleaning in which at least a two-way operation is applied with the washing solution: it is first subjected and then moved out by one or more devices (e.g. pipette, sampling pump etc). The two-way cleaning is commonly used in the present chip testing. For example, the two-way cleaning in cleaning the reactor with remnant sample involves two-way operation, namely moving the reaction residual out of the reactor, and then subjecting and removing of the washing solution to and from reactor. The two-way operation usually is repeated several times, somewhat like the washing process in the ELISA testing.

The term “one-way cleaning” refers to a reactor cleaning in which only an one-way operation is applied with the washing solution. For instance, the one-way cleaning of the reactors containing remnant samples is conducted by spurting the washing solution directly onto the reactors so as to move the residuals out of the reactors. In another case of one-way cleaning, the removal of remnant samples and the washing of reactors are conducted at the same time when washing solution is subjected.

The term “cleaning system” refers to all the hardware and software in the testing device, which is used to the reactor cleaning, especially the cleaning of reactor containing reaction residuals, such as the cleaning system mentioned in the implementation of this invention. The term “fluidity-decreasing system” refers to all the hardware and software in the testing device, which is used to decrease the fluidity of the reaction residuals in the reactors. The term “sample spotting system” refers to all the hardware and software in the testing device used to the sample subjecting by spotting.

II. Application Patterns of Invention

Prior to go to the details of this invention, it has to be understood that this invention is not limited to the fixed application pattern (such as certain fixed process, parameters and combinations), for despite the definite content of invention, the procedures or parameters are variable. It is also to be understood that the terminology used herein is for the purpose of describing particular embodiments of the invention only, and is not intended to be confining.

This invention provides a testing device of analytic chip of multiple reactors, which comprises: a). one-way-cleaning system for one-way-cleaning of remnant sample in said reactor, wherein said one-way cleaning comprises subjecting Q individual fluids to N reactors respectively, by which said remnant samples are washed away from said reactors, wherein Q≧N≧2; or/and b). fluidity-decreasing system for fluidity-decreasing of remnant sample; or/and c). sample spotting system for sample subjecting by spotting.

A testing device of this invention comprises the one-way-cleaning system by which the reactor with the remnant sample is cleaned through the one-way cleaning. By now, one well-established principle to avoid the cross-contamination among the multiple reactors on a chip in reactor cleaning is to have absolute physical isolation, that is, to completely prevent the sample, reaction residual and the washing solution containing the residual in one reactor from entering another reactor. The present two-way cleaning is an exemplification based on this principle. Amazingly, in the implementation of this invention, cross-contamination can be avoided by the one-way cleaning of the device of this invention, which does not require absolute physical isolation. With no intention of theoretic discussion, we just offer an example to make the point clear:

When the washing fluids A and B, from outlet A and the adjacent outlet B of a nozzle correspondingly, reach the probe regions A and B on the reactors A and B respectively, for the hydrodynamic reasons, each splash thereby produced can hardly reach the probe region covered by another washing fluid even if it enters another reactor. In addition, even if small amount of splash A does enter the probe region B. no significant reaction may occur, due to chemical dynamic reasons, because: a). a very low concentration of possible remnant sample A, is greatly diluted not only by the fluid A in the reactor A, but also by the fluid B in reactor B; b). the continual flowing of washing fluid B makes a very short contact between the probe region B and the possible remnant sample A in the splash. Therefore the risk of cross-contamination among reactors is still under control based on the hydrodynamic and chemical dynamic isolation in this invention even without absolute physical isolation (e.g. reaction residual A may enter the adjacent reactor B). For instance, even though reaction residual A enters the adjacent reactor B, no significant reaction will take place between residual A and probe array B. Also, the said one-way cleaning is not only applicable to the reactors with remnant sample but also to the reactors with some other reaction additives (e.g. remnant markers).

Compared with the device with the two-way cleaning system such as the ELISA device, the device with the one-way cleaning system of this invention is simpler, more economical and more practicable in the process of automation. Besides, since the removal of residuals is faster and more complete, it is more convenient, timesaving (e.g. with less than 10 seconds) and effective (with less nonspecific label left), which ensures a higher sensitivity.

A testing device of this invention comprises the sample spotting system by which the sample subjecting is performed through the sample spotting. In this invention, the sample subjecting by spotting includes the sample subjecting by the contact spotting or/and by the non-contact spotting. At present, the spotting is used only for the probe immobilization on the reactor. The said sample spotting in this invention is used not only for the sample subjecting on the reactor, but also for subjecting other reaction additive (e.g. marker, etc.) onto the reactor. Here the sample refers to that to be tested.

According to the Implementation of this invention, the sample spotting of this invention has the following advantages, compared with the present sample subjecting (e.g. sample injection):

a). it can better control the amount of sample, and particularly the area covered by liquid sample on the bottom of the reactor, consequently it can better control the cross-contamination in the reactor cleaning especially by the cleaning method introduced in this invention;

b). mechanization and automation are more accessible since spotting tip (e.g. a tip with solid center or/and hollowed tip or/and water absorbent material) is employed instead of other mechanic inlet/outlet devices, which can easily control the size of sample spotted.

c). it can optimize the sample distribution on the probe region by applying a kinetic energy, so that probe spot can have a better reaction with substance of interest. As a result, a higher sensitivity can be achieved.

A testing device of this invention comprises the fluidity-decreasing system by which the remnant sample is treated through decreasing its fluidity. The fluidity decreasing of the remnant sample will limit its movement, consequently it reduce the cross-contamination risk. At present, no similar remnant-sample treatment is applied in the testing of chip of multiple reactors.

In certain aspects, the testing device of this invention includes any combination of the following systems of this invention: a). the sample spotting system, b). the fluidity decreasing system, and c). the one-way cleaning system. Some examples of the combination are: A). testing device including the above systems a) and b); B). testing device including the above systems a) and c); C). testing device including the above systems b) and c); D). testing device including the above systems a), b) and c).

In a testing device according to the testing device of this invention, said Q fluids are subjected respectively to said N reactors at almost the same time. In this invention, the expression “arriving almost at the same time” can be described by the arriving-time difference. In this invention, the term “arriving-time difference” refers to the time difference between the one of the Q fluids that first arrives at one of the N reactors and the one of the Q fluids that last arrives. For example, when N independent fluids spurt to N reactors, if the first fluid arrives at 9:55′55″, while the last fluid arrives at 9:56′05″, then the arriving-time difference is 10 seconds. For example, if the arriving-time difference is less than 10 seconds, preferably less than 3 second and optimally less than 1 second, we may think that they are two fluids “arriving almost at the same time”. This parameter is significant to the testing method in this invention (table 1).

TABLE 1
Relation between the difference of arrival time of various
washing fluids (DAT) and the occurrence of the cross-contamination
among reactors (OCR) in 20 assays

| assay* | DAT (second) | OCR |
|---|---|---|
| 1 | 20 | >1 |
| 2 | 10 | 0 |
| 3 | 5 | 0 |
| 4 | 3 | 0 |

*the assays are performed under the conditions similar to that in the implementation 8.

As an example of application of the device of this invention, a testing method may comprise at least the following steps: a). providing an analytic chip with M reactors and getting N reactors of them ready for use, wherein said reactor number M≧N≧2; b). providing samples probably containing substance of interest and getting them ready for use; c). combining said sample of the step b) with said probe spot in the reactor of the step a) under conditions which allow said substance of interest to bind to said probe spots; d). getting N said reactors ready for washing after the binding reaction of the step c), wherein said reactor contains remnant sample; and e). subjecting Q individual fluids into said N reactors of the step d) for the washing, wherein: (i). said remnant sample is removed by spurting of said fluid; and (ii). said fluid number Q≧said reactor number N. Wherein:

An example for the expression “providing an analytic chip of M reactors and getting N reactors ready for use” is to provide a chip with M protected reactors and then remove the protective structure of the N out of M reactors of the chip so as to make it ready for sampling, wherein the chip provided can one or several.

An example for the expression “providing samples probably containing substance of interest and getting them ready for use” is to prepare samples probably containing substance of interest for loading through certain procedures such as dilution, concentration or/and labeling.

An example for the expression “combining said sample of the step b) with said probe spot in the reactor of the step a) under conditions which allow said substance of interest to bind to said probe spots” is to subject the prepared sample into the reactor ready for use and make the probe react with the probably existing substance of interest. The said sampling can be conducted in way of spotting in this invention.

An example for the expression “getting N said reactors ready for washing after the binding reaction of the step c), wherein said reactor contains remnant sample” is to get the N reactors with the remnant sample ready for washing right after the said reaction in case of open reactor, or to get N reactors ready for washing in case of closed reactors by two processes: (a). removing part of the remained remnant samples; and (b). removing the closed structure above the probe region.

An example for the expression “subjecting Q individual fluids into said N reactors of the step d) for the washing, wherein: said remnant sample is removed by spurting of said fluid” is to spurt N separated airflows or washing-solution fluids to the corresponding N exposed reactors and wash away the remnant samples by splashing.

In a testing device according to the testing device of this invention, said one-way-cleaning system comprises nozzle with Q outlets by which said Q fluids are produced. Though some other cleaning systems may be equipped with nozzles, they are designed to clean the reactor of the remnant marker, with which the said washing mentioned in the testing method of this invention cannot be conducted. Therefore, they are not of the features of the said cleaning system in the invented device.

In a case of the testing device with the cleaning system of this invention, the said nozzles are located in correspondence with the said exposed reactors of said chip of multiple reactors.

In said testing device with cleaning system, the said cleaning system may also comprise one or more of the following components: a. the flowing-direction controlling system of said flow at the outlet of said nozzles; b. the mechanism controlling the relative distance between said nozzle and said substrate immobilized with the probes (e.g. the all-around nozzle and/or the support of chip); c. ultrasonic convertor; d. the enclosed chamber which prevents reaction media and the said washing solution from polluting environment; e. fluid dynamic system, etc. The said fluid dynamic system includes the pump controlling speed and pressure of fluid or airflow, canal and fluid speed/pressure controlling system that determines the pattern of the fluid (e.g. the starting time, duration and whether it is in continual or pulse flow).

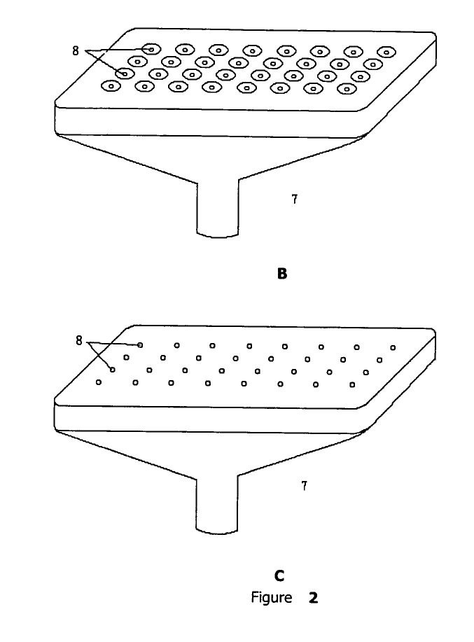

In said testing device with cleaning system, the said nozzle is of diverse types, such as the protruding nozzle with outlets located on the top of a convex body (FIG. 2A), the depressed nozzle with outlets located at a concave (FIG. 2B), the plane nozzle with outlets on the plane (FIG. 2C) and their combinations. In the reactor cleaning process, the fluid may be arranged in the pulse mode, continual mode, water-gas mixed mode or the combined mode. The nozzles and/or chips are movable, either circling or oscillating, so as to guarantee an even wash.

In a case of the said testing device with cleaning system of this invention, various nozzles are available for diverse analytic chip of multiple reactors or for different use.

In said testing device with cleaning system, the said cleaning system can be used not only to clear the reactor of the remnant samples, but also to clear the reactor of other reaction residual such as remnant marker. In said testing device with cleaning system, different chips with multiple reactors can be used, but the analytic chip of multiple reactors based on plane substrate is preferred.

Through the implementations of this invention, we find that the said cleaning system will not cause cross-contamination among multiple reactors, even the densely located reactors on the chip. Besides, it has a better washing result.

In a testing device according to the testing device of this invention: a). linear speed of said fluids arriving on said reactors is 1-1 000cm/second, preferably 100-500cm/second; or/and b). diameter of said outlet is 0.1-1.0 mm; or/and c). density of said outlets on said nozzle is over 1 unit/cm²; or/and d). clockwise angle between said individual fluid and said probe region is 1-179 degrees; or/and e). distance between any of said outlets and its nearest probe region of said reactor is 0.1-10.0 cm. In addition, the spurting speed of the fluid is also an important parameter, correlated to the designed reaction data, such as the size of probe array, the spacing between probe arrays etc. In a word, to decrease the risk of cross-contamination is a chief decisive factor among all these parameters.

In a preferred one-way cleaning system of this invention, the said Q fluids reach the N reactors with approximately same momentum, especially the same linear speed. The said linear speed is changeable or not (e.g. in continuous change or pulse change) in the reactor cleaning. And with the said testing method, the linear speed of said washing fluid that first reaches the immobilized probe is high enough to help minimize the cross-contamination among reactors and enhance the purification of reactors. As indicated in Table 2, a proper linear speed is also significant to alleviate the interference of background noise. In Table 2, the specific background noise spot refers to the background spot whose size is no less than that of probe spot and whose signal intensity is no less than 50% of that of positive sample.

TABLE 2
Relation between linear speed (LS) and the frequency
of specific background noise (FSBN) in the 20 assays.

| assay* | LV (cm/second) | OSBN |
|---|---|---|
| 1 | 5 | >10 |
| 2 | 10 | 7 |
| 3 | 20 | 5 |
| 4 | 100 | 0 |
| 5 | 200 | 0 |

*the assays are performed under conditions similar to that in the Implementation 8.

One thing noticeable is that the linear speed is produced by pressure (such as pump or compressed air). In a one-way cleaning of the reactor with the remnant of this invention, the said fluid may also present other physical powers including ultrasonic power.

The clockwise angle between the fluid-spurting direction and the probe region surface on the reactor can be set preferably at 90±5 degrees (spurting upward or spurting downward), either by manual adjustment or the angle adjustor set within or outside of the said cleaning system. The spacing between the outlet and the corresponding probe region can be adjusted by adjusting the position of the nozzle or/and the chip. In our research, no cross-contamination is observed in all of the above parameters.

In a testing device according to the testing device of this invention, said fluidity-decreasing system consists of at least one of the following systems: a). temperature controlling system or/and moisture controlling system used to increase viscosity of said remnant sample; b). loading system for subjecting fluidity-decreasing additive into said remnant sample; and c). water-absorption system, comprising water absorbent, for decreasing volume of said remnant sample.

Since at present, all the remnant samples are moved out of the multiple reactors by sucking, the presently existing cleaning system is not expected to increase the viscosity of the residual. As a result, though presently existing devices may have a temperature controller or/and moisture controller, it is only for the reaction not for the reactor cleaning. Sometimes their objective condition (no obvious increase of viscosity for the reaction media in the reactors) is contrary to that of our invention (evident increase of viscosity for the reaction media in the reactors).

Besides, though the presently existing devices may also contain the additive loading system, it is only for adding the liquid media and will not decrease the fluidity of media. While in this invention, the additive loading system is designed to add the fluidity-decreasing additives (preferably in form of powder) so as to decrease the fluidity of the reaction media. Of course, the temperature controller, moisture controller or/and sampling system in this invention can also serve some other purposes, while without changing its own characteristics.

In fact, a lower fluidity will help restrict the flowing of the remnant samples and hence control the cross-contamination. With the present chip testing, the remnant sample should be of a larger volume so that it can be removed easily. Consequently, any attempt to increase fluidity of the remnant sample or quantitatively decrease the volume of samples or reaction residual after sampling should be excluded. However, in the implementation of this invention, we find that when the viscosity of reaction residual is increased to the extent (e.g. by over 30%) that its fluidity is deprived, the splashing in the reactor cleaning is better controlled, which will help minimize the cross-contamination risk. In the implementation of this invention, the fluidity of the remnant samples is also decreased by additive, which increases viscosity or/and restricts the flow of the remnant samples. The additive used in this invention is chemically/biologically stable additive, including carbohydrate, polymer powder, chromatography gel, particle with multiple pores etc. For instance, the fluidity of the remnant samples is decreased by decreasing volume of said remnant samples by water-absorbing substance (e.g. paper fibers).

In a testing device according to the testing device of this invention, said sample spotting system includes one or more contact-spotting tips for subjecting sample to said reactor by the tip contacting. An optimal design of the said contact tip in this invention should include solid tip (e.g. needle tip, cast-shaped tip etc.) or/and hollowed tip (e.g. capillary tube etc.) or/and water absorbent tip (e.g. fiber stick, paper stick etc.). The contact-spotting tip refers to the devices such as capillary tube, needle or cast, which can transfer sample to reactors or to probe region, without any external mechanism (e.g. injector in the injection sampling). The optimal contact-spotting tip is the one based on capillary phenomenon or/and hydrophilic adsorption or/and water adsorption. An example of the contact-spotting tip is a cast-shaped tip with slop, wherein: a). the diameter of cast is over 1 mm; b). one or more slops are on the bottom of the cast with the width of 30-200 μm. When several spotting tips are put into the multi-well plate filled with different samples, the micro-structures on the cast (e.g. slop, tip etc.) will help adsorb the samples and distribute them by surface tension. An optimal design of the said contact tip in this invention should include solid tip (e.g. needle tip, cast-shaped tip etc.) or/and hollowed tip (e.g. capillary tube etc.) or/and water absorbent tip (e.g. fiber stick, paper stick etc.). The said spotting tip can be made of different materials (e.g. metal, plastic, glass etc.)

An example of the said sample spotting system is featured by: a). the sample spots are surrounding the center of probe region and the nearby area. b). the spotting tip should be big enough so that the area covered by sample spots on the bottom of reactors should be 1.5-5.0 times of the area of probe region; optionally c). the spotting tip should drop at such a speed that the linear speed of samples subjected to probe region is over 0.1 cm/second.

In a testing device according to the testing device of this invention: a). linear speed of said sample spotted to said reactor is over 0.1 cm/sec; and/or b). sample spot formed by said contact spotting covers an area on said reactor, which is 1.5 to 5.0 times as large as that of probe region of said reactor.

The sample spotting system of this invention is different from the sample injection system adopted in the present testing method of chip of multiple reactors, which concerns the volume of sample only instead of the controlling of the position, size and momentum. In a preferred sample subjecting by contact spotting of this invention, only a minimized volume of the sample is spotted onto the reactor, which needs only a minimized diameter of the fluid in the one-way cleaning of the reactor with remnant sample. This will help minimize the splashing of the spurted fluid containing the remnant sample, and consequently minimize the cross-contamination risk in the reactor cleaning. In a preferred sample subjecting by contact spotting of this invention, 1-10 μl, preferably 1-3 μl sample is spotted onto the reactor; and a sample spot with an area of 1-36mm², preferably 1-16 mm² is formed on the reactor.

A testing device, according to the testing device of this invention, also comprises optical-signal detecting system, wherein: a). said detecting system contains background-signal intensifying system which comprises light radiating or/and reflecting structure in background area of detected chip; or/and b). said detecting system contains background-signal weakening system which comprises light absorbing structure in background area of the detected chip, whose light absorbency is over 95%.

In the device of this invention, the said chip does not require a high partition structure. Instead, in most cases, the lower the partition structure is, the better cleaning and/or spotting is. In this invention, an optimal chip has a low partition structure (with a height of less than 1 mm, preferably less than 0.5 mm and optimally less than 0.3 mm). However in the two-way cleaning now in use higher partition structure (e.g. ELISA plate washer) is preferred, usually over 1 mm, even over 3 mm. In this invention, the said analytic chip refers to any chip of multiple reactors, including those mentioned in this invention, and analytic chip of multiple reactors now available in market and in some inventions (e.g. PCT/CN03/00055 and PCT/CN2004/000169).

In this invention, the said chip of multiple reactors includes the chip of multiple open reactors, preferably the chip with multiple open non-flow reactors, optimally the chip with multiple open non-flow plane reactors. In this invention, the said chip also includes the chip with multiple closed reactors, preferably the chip with multiple reversibly closed flow reactors, optimally the chip with multiple reversibly closed reactors containing spacious-chambers, such as the chip with multiple reversibly closed reactors containing wet chamber in PCT/CN2004/001128. The said device in this invention can be applied on not only one analytic chip but also on several chips in parallel.

A testing device, according to the testing device of this invention, also comprises analytic chip of multiple reactors, composed at least of substrate, probe spot immobilized thereon, and reactor partition structure linked thereon, wherein at least part of said partition structure presents a height less than 1 mm. In the said chip, the substrate is of activated or inactivated glass, plastic and metal, such as the activated glass slide containing one or more of the following derivative groups: amino-, aldehyde, —CO—NHNH₂, H₂N—NH—CONH—, DEAE-, QAE-, CM-, SP-, MEP-, siloxane, thiol-.

In a testing device according to the testing device of this invention, said partition structure comprises hydrophobic structure and/or hydrophobic-lipophobic structure, wherein said partition structure comprises hydrophobic-lipophobic structure whose surface is more hydrophobic and lipophobic than that of said substrate. The first chip of this invention, in which the partition structure presents a very limited height (e.g. less than 0.50 mm), is different from the present chips with partition structure based on height-difference between the substrate and the partition structure, in which the partition structure often presents a height of over 1.0 mm. Though the partition structure based on height difference may hold the water medium, oil medium and hydrophilic-lipophilic medium from moving to other reactors in the course of sampling, reaction and even the course of residual washing, the hydrophobic-lipophobic partition structure in this invention is not restricted by the height difference. It is not only accessible to some scanners (such as fluorescent scanner) but also more applicable in the testing method of this invention.

In a first chip of this invention: a). said surface of hydrophobic-lipophobic structure presents a water contact angle 40 degrees, preferably 80 degrees more than that of said substrate; and b). said surface of hydrophobic-lipophobic structure presents an oil contact angle 10 degrees, preferably 40 degrees more than that of said substrate. In fact, the bigger the water contact angle of the said hydrophobic-lipophobic structure is than that of the said substrate, the stronger it is to limit the unnecessary moving of aqueous solution, while the bigger the oil contact angle of the said hydrophobic-lipophobic structure is than that of the said substrate, the stronger it is to limit the unnecessary moving of organic solution and the more antifouling it is.

In a first chip of this invention, the said hydrophobic-lipophobic structure comprises hydrophobic-lipophobic material. Though the hydrophobic-lipophobic structure in the invented chip can be made in different patterns, such as the hydrophobic-lipophobic nano-structure made by mechanical processing, the optimal component is hydrophobic-lipophobic materials. The hydrophobic-lipophobic material is combined with the substrate by direct linking (e.g. coating, painting, adhering, etc.) or indirect linking (e.g. linking hydrophobic-lipophobic material with substrate by a third party). This is one example of the indirect linking: the hydrophobic-lipophobic material is painted to the corresponding part on the forming plate, which is then adhered to the substrate immobilized with probe array, or is adhered to the substrate before the substrate is immobilized with probe array.

In a first chip of this invention, the said hydrophobic-lipophobic material includes hydrophobic-lipophobic organic material or/and hydrophobic-lipophobic nano-material. Some examples of these hydrophobic-lipophobic organic material and hydrophobic-lipophobic nano-material are given in the implementation examples of this invention.

In the first chip of this invention, the said reactor partition structure includes hydrophobic-lipophobic coating on the substrate; hydrophobic-lipophobic convex body on the substrate (e.g. hydrophobic-lipophobic adhesive tape) and forming plate with the hydrophobic-lipophobic structure, wherein the said forming plate is either reversibly or irreversibly linked with the said substrate; the said forming plate might be a plate of macromolecular material, textile plate or metal plate etc, e.g. a plastic forming plate coated with the hydrophobic-lipophobic materials or a forming plate of the hydrophobic-lipophobic plastic.

The said forming plate also can comprise other structures, such as one or more of the following structures which can control the speed of reactive medium flowing in a certain direction: the hydrophilic coat, hydrophobic coat, absorptive coat based on capillary phenomenon and the draining pipeline, bucket and strip that help control the flowing. In the invented analytic chip of multiple reactors, the surface of chip is larger than that of substrate, that is, part or all of other structures of the reactors (such as inlet and/or outlet structure) are set outside of the substrate on the forming plate.

In a first chip of this invention, the reactors are covered by protective structure when they are not in use. The said protective structure covers at least part structure of reactor when no sample is to be loaded, while part or all of it can be either reversibly or irreversibly dismantled in the process of sampling.

The first kind chip of this invention includes the analytic chip of multiple open reactors or the chip with multiple closed reactors. The first chip with multiple closed reactors include chip with multiple reversibly closed reactors, which is composed of substrate, probes immobilized thereon, partition structure linked to substrate and forming plate. The partition structure linked to substrate contains the said hydrophobic-lipophobic structure, while the partition structure on the forming plate may or may not contain the said hydrophobic-lipophobic structure.

A first chip with multiple reversibly closed reactors of this invention is composed of two parts: part I consisting of the substrate, the probe immobilized on the substrate, the hydrophobic-lipophobic partition structures linked to substrate; and part II, the forming plate which can be partially or totally removed when washing the reactors. When samples are added, the forming plate and other structures (including substrate and the probes on it as well as partition structure among reactors) can form multiple reversibly closed reactors through reversible conjugation. Examples of the said reversible conjugation include the conjugation based on one or more of the following forces: the mechanic forces produced by gravity, elasticity, screw or fixturn, the magnetic force generated by magnet or electrolyzer and removable adhesion provided by adhesives. When the said exposed reactors are needed, the said reversible conjugation can be canceled, e.g. by removing the magnetism produced by magnetic fixturn etc. One example of multiple reversibly closed reactors is multiple reversibly closed reactors with spacious chamber.

In a chip of the testing device of this invention, said reactor contains non-probe region, and probe region that presents a minimized area; and at least part of said partition structure presents a surface that is more hydrophobic than that of said substrate. The second kind of chip of this invention can be used as a part of the invented testing device, especially the testing where the volume of samples loaded is minimized.

The second chip of this invention is different from the existing chip of multiple reactors. The existing chip only presents characterized partition structure, while the second chip of this invention presents not only characterized partition structure but also the characterized probe region. The latter plays an important role in fighting against cross-contamination among reactors. In fact, the smaller area of the probe region is, the less samples can be subjected in a reactor, and the smaller risk of cross-contamination is (table 3).

TABLE 3
Results of anti-cross-contamination test
of biochips with different structures

| assay * | PRA ** | PSH *** | PSSCA **** | Testing ***** | FCR |
|---|---|---|---|---|---|
| 1 | 0.9 mm² | 0.06-0.20 | 113° | − | 0 |
| 2 | 0.9 mm² | 0.06-0.20 | 78° | − | 0 |
| 3 | 1.6 mm² | 0.06-0.20 | 113° | − | 0 |
| 4 | 1.6 mm² | 0.06-0.20 | 78° | − | 0 |
| 5 | 4.0 mm² | 0.06-0.20 | 113° | − | 0 |
| 6 | 4.0 mm² | 0.06-0.20 | 78° | − | >1 |
| 7 | 9.0 mm² | 0.06-0.20 | 113° | − | >1 |
| 8 | 9.0 mm² | 0.06-0.20 | 78° | + | >1 |

* the assays are performed under conditions similar to that in the Implementation 9.
** PRA- probe region area
*** PSH- partition structure height
**** PSSCA- water surface contact angle of partition structure
***** Testing: when said chip is rotating 0-360°, if distilled anionic water sample subjected to the probe region will not spill out of any of said reactors, the result is negative (−), otherwise the result is positive (+), wherein: (i). The rotating speed is 36°/second; (ii) the volume of said water sample (V) = the bottom area of said probe region × (minimum height of said partition structure + 100 μm).

With the minimized probe region, not only the amount of sample can be minimized but also fewer amount of the probe will be consumed.

In a second chip of this invention: a). said minimized area is less than 4.0 mm², and is smaller than the area of said non-probe region; or/and b). said height of partition structure ranges from 0.01 mm to 0.80 mm; or/and c). said surface of partition structure presents a water contact angle 40 degrees more than that of said substrate surface.

In a second chip of this invention: a). said minimized area is less than 1 mm²; and/or b). said probe region presents a density of more than 9 probe spots/mm².

In a second chip of this invention, said area of probe region, said height of partition structure and said water contact angle are determined in this way that when said chip is rotating 360°, no water sample subjected to said probe region will spill out of any of said reactors. This testing is performed under the following conditions: the rotating speed is 360°/second; (ii) the volume of said sample (V)=the bottom area of said probe region×(minimum height of said partition structure+100 μm).

This preferred second chip comprises not only the characterized partition structure and characterized area of probe region, but also the specific relation between the area of probe region and partition structure. In fact, the minimized probe region (able to minimize the volume of the sample loaded), the low partition structure (helpful for the application of this invention) and the maximized hydrophobicity of the partition structure (effectively avoiding the unnecessary movement of water solution) combine into a unified structure against cross-contamination, which may make the risk of cross-contamination controllable in the testing.

In a second chip of this invention, said partition structure includes: a). hydrophobic structure whose surface presents a water contact angle between 20-59 degrees more than that of said substrate surface; or/and b). superhydrophobic structure whose surface presents a water contact angle 60 degrees more than that of said substrate surface; or/and c). the hydrophobic-lipophobic structure mentioned above.

The second kind chip of this invention includes the following analytic chips: the chip of multiple open reactors, the chip with multiple closed reactors, the chip with multiple reversibly-closed reactors, the chip with multiple flow reactors and the chip with multiple non-flow reactors etc.

In a second chip of this invention, the substrate is a plane substrate. In the second kind of chip of this invention, the said partition structure includes one or more following structures: coating on the substrate; convex body on the substrate (e.g. adhesive tape); forming plate reversibly or irreversibly linked with the substrate, etc. In the second kind of chip of this invention, the said forming plate can also be made of other structures, such as one or more of the following structures which can control the flowing: the hydrophilic coat, hydrophobic coat, absorptive coat based on capillary phenomenon etc. In the second kind of chip of this invention, the surface of chip is larger than that of substrate, where part or all of the reactors structures are set on the forming plate outside of the substrate.

As shown in the implementation examples of this invention, the analytic chip of the invented device presents some features in its minimum composition (substrate, probe spot immobilized thereon, reactor partition structure linked thereon) and its height (at least part of the partition structure less than 1 mm). The said analytic chip of multiple reactors is intended to achieve the following goals:

The said analytic chip is highly integrated, that is:

its structure is highly integrated, for instance, a chip of 25×75 mm(width×length) may contain several reactors (e.g. 16-48 or even more reactors)

its application is highly integrated, for instance, it can be applied to the highly integrated testing of this invention.

The said analytic chip is highly efficient, for instance, the reactors on it are arranged in high density (e.g. 1-2 or more reactors/cm²). Thus it helps reduce the testing cost and is more applicable in the highly integrated testing.

3). The said analytic chip is very safe, for instance, no cross-contamination among reactors is observed in the implementation of this invention.

Though the invented device is preferred in the chip of multiple reactors, it can also be adopted in the analytic chip of one reactor. With the invented device, manipulation can be conducted on one chip of multiple reactors or simultaneously on several chips of multiple reactors; manipulation can be conducted either on part of the reactors or all of the reactors on a chip.

The invented device may be composed only of the said cleaning system, or cleaning system and fluidity-decreasing system, or cleaning system and other systems which help accomplish the chip testing such as sample preparation system, sample loading system, reaction system, marker preparation system, marker loading system, signal detecting and analyzing system, etc. Like many other devices, different functional system consists of either different or same units, apparatus and components.

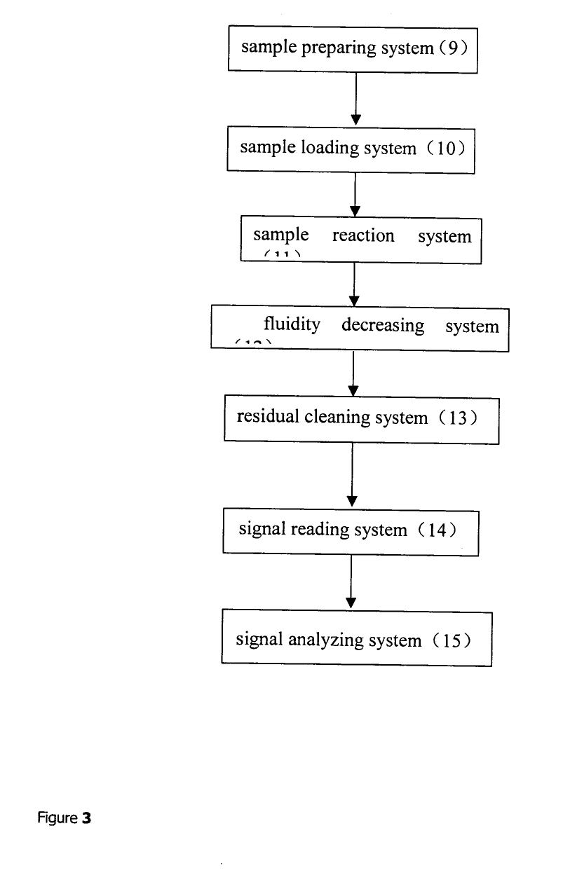

The following example will illustrate the procedure and the logic relations among different systems of a testing device of this invention (FIG. 3):
1. Sampling system (10) is used to transfer the samples (e.g. diluted sample, or the samples with marker) prepared by the sample preparation system to the N reactors of the chip (e.g. such protective structures as a protective coat covered on the open chip is opened);
2. The reaction system (11), such as chip incubation box, the reaction system with temperature/moisture controller, will provide the condition required for the samples to react in the reactors;
3. After the reaction, viscosity increasing system (12), e.g. temperature/moisture controller, is applied to increase the viscosity of the remnant samples; or/and sample loading system is used to add fluidity-decreasing addictives, so as to decrease or eliminate the fluidity of the residual;
4. cleaning system (13) is used to wash the reactors;
5. If necessary, marker loading system (e.g. pipette or the sample loader with the micropump) is adopted to add the markers prepared by marker preparing system to the multiple reactors, which are cleaned and dried. The marking reaction will take place under the condition manipulated by the reaction system, after which the cleaning system is used to clear the remnant samples and wash the reactors simultaneously;
6. With the help of signal detecting and analyzing system, the testing signal in the chip-reactors is detected and analyzed.

The invented devices refer to the devices either capable of all detecting functions or of some detecting functions, which can be used to conduct all the testing procedures mentioned in step A-F or only part of the procedures such as step C and D, or step C to F. In the invented testing device, a central controlling system controls the systems with different testing functions. And the more functions the device has, the more efficient it is, which is also true if the testing device contains only part of the above functional systems.

In addition, the same component in the testing device of this invention can be applied to different functional systems. For instance, in one of our implementations, some components in said cleaning system can be employed either to remove remnant samples or to remove the remnant marker. The removal of remnant markers and washing can also be achieved by other means, such as the nozzle with single outlet or the nozzle with single row of outlets etc.

Implementation

The following implementations of this invention and figures will illustrate this invention in more details. Though they are only some cases of this invention, the professional can naturally deduce the other implementations of this invention based on these examples.

Implementation 1: A Testing Device Containing a Reactor-Cleaning System for Testing of Analytic Chip of Multiple Reactors

The testing device prepared in this example contains a reactor-cleaning system that is composed of the following subsystems:
a. Fluid system for producing and transporting the fluid, including: storing bottles for washing solution, electromagnetic valves, pump, pipelines and nozzles with multiple outlets etc.
b. Sewage system for discharging the sewage, including: some drainage pipelines, pool for the liquid waste etc.
c. Anti-leaking system, including one separate chamber for the reactor cleaning, which can be opened or closed at will.
d. Controlling system, which controls pressure/speed of the fluid, temperature, position of the nozzle and position of the chip (angle adjustor).
e. A central controller in charge of coordination among subsystems.

The testing device in this example can either include a reaction controlling system for controlling or not.

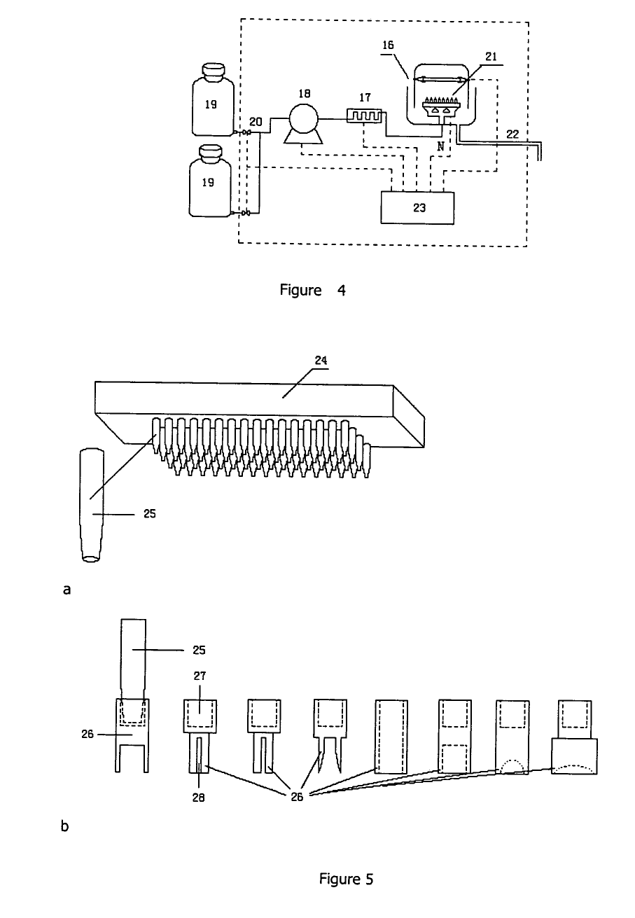

The testing device in this example, comprising reactor-cleaning system and reaction controlling system, works in the following way (FIG. 4):

First, open the separate washing chamber (16), place the chip with N (N>1) open reactors loaded with sample into separate washing chamber at the optimal angle, and then close the separate washing chamber. Secondly, adjust the temperature/moisture as required by the temperature/moisture controller (17). After the reaction is completed within the required time, turn on pressure pump (18) to press through the pipeline (20) the washing solution at the optimal temperature in the storing bottle (19) to the optimal nozzle with N outlets in the optimal position. As a result, each of the N separate washing fluids will spurt to each probe region of the N reactors of the chip simultaneously from N outlets with optimal pressure/speed. These separate washing fluids will splash around, washing away the remnant samples from the reactor. All the splashing liquid will go into the pool for the liquid waste through the outlet path (22). After that, the nozzles with optimal rotation will go on to wash other regions of reaction well until the washing is over. At last, chip will be dried with wind from an air-blower at 25-30° C., and then it is taken out of the separate washing chamber.

The working parameter for the cleaning system in the testing device and its changing range should be set so as to meet the need of practical testing. In this implementation, the optimal angle of the chip in the separate washing chamber is adjusted by the angle adjustor connected with the chip, ranging from 0 to 180°. The optimal temperature of the separate washing chamber is adjusted by a temperature controller, ranging from 20-40° C. The pressure/speed of washing fluid at the outlet is controlled by fluid pressure/speed controller, with pressure ranging from 0-7 kg/cm² or speed ranging from 10-1000m/min/mm². The optimization of the nozzle is achieved by replacing nozzles. Parameters of the nozzles prepared in this example are listed in table 4. The optimal position of the nozzles means the optimal spacing between the outlet of the nozzle and the probe region in reactor, which is adjustable between 0 mm and 30 mm by the distance adjustor connected with nozzle. The optimal rotation of the nozzles is provided by a rotator, with the rotating speed of 1-5 round/second, the rotating diameter of 4 mm.

The testing device prepared in this implementation can either comprise a marking system or not. The said marking system consists of storing bottle for marker solution, a pump to add marker solution, an injector of marker solution etc. The testing devices prepared in this implementation, which consists of the cleaning system, the reaction system and the marking system, works in this way:

After the reaction and the reactor cleaning of the remnant sample is finished as mentioned above, turn on the pump to transfer marker solution in the storing bottle at optimal temperature to the injector of marker solution, which will inject the optimal amount of marker solution to the said N dried reactors following the preset route. At the same time, set the temperature/moisture of the place where multiple reactors are located as required by reaction. After the marking reaction is completed within the required time, clear the reactor of remnant markers following the same working principle as in the washing of the remnant sample.

All the devices in this implementation are of common preparation. The above angle adjustor, nozzles, separate washing chamber, distance adjustor, etc. are made in a mechanic processing plant, while other components, auxiliary parts and controlling devices are available on the market.

TABLE 4
parameters for nozzle with multiple-outlets

| | | Number of outlets | |
|---|---|---|---|
| nozzle | Diameter of outlet | (N) | Density of outlets |
| 1# | 400 μm | 56 | 3.7 unit/cm² |
| 2# | 500 μm | 32 | 2.1 unit/cm² |
| 3# | 600 μm | 24 | 1.8 unit/cm² |
| 4# | 700 μm | 16 | 1.0 unit/cm² |

With the one-way cleaning system in this implementation, washing can be simultaneously conducted on several reactors (e.g. 16-48 reactors), especially the reactors with high density (e.g. with a density over 1 reactor/cm²). It usually takes a very short time (e.g. 1-10 seconds) and produces a better washing effect as the linear speed of washing fluid is increased. Besides, the cross-contamination is also controllable in the washing.

Implementation 2: A Testing Device Based on Analytic Chip of Multiple Reactors Containing a Fluidity-Decreasing System

The testing device of chip of multiple reactors prepared in this implementation contains a fluidity-decreasing system.

The said fluidity-decreasing system prepared in this implementation refers to the fluidity-decreasing system by temperature/moisture controlling and the fluidity-decreasing system by fluidity-decreasing additives, wherein the former includes temperature/moisture controlling system (comprising temperature/moisture controllers etc.), while the latter includes fluidity-decreasing additives loading system.

In this implementation, temperature and moisture are limited to 15-45° C. and 40-95% respectively by the temperature/moisture controller used for controlling reaction and decreasing fluidity. The said fluidity-decreasing system on the basis of temperature/moisture controlling works in this way: by adjusting the temperature and moisture of the area where chip is located, we can control the water evaporation in the medium, thus controlling the viscosity of the medium containing water and consequently the fluidity of the medium containing water.

In this implementation, the fluidity-decreasing system based on fluidity-decreasing additives comprises the powder sprayer whose outlet is directed to the separate washing chambers. The powder of fluidity-decreasing additives will be spayed to the chip when necessary, which will be brought to the well for liquid waste by the washing solution after contacting with remnant samples. The said fluidity-decreasing system based on fluidity-decreasing additives works in this way: by subjecting the powder of fluidity-decreasing additives, we can increase the viscosity of the medium containing water or/and decrease the freely flowing liquid in the reactors so as to control the fluidity of medium containing water.

In this implementation, all the devices in use are the common devices, wherein the temperature/moisture controllers are selected from the products available on market, while other components and auxiliary parts can be obtained as in the implementation 1.

With the fluidity-decreasing system in this implementation, the fluidity controlling of liquid media can be simultaneously conducted on several reactors (e.g. 16-48 reactors), especially the reactors with high density (e.g. with a density over 1 reactor/cm²). The whole process usually takes a very short time (e.g. 1-10 seconds) and can effectively decrease the fluidity of remnant sample.

Implementation 3: A Testing Device Based on Analytic Chip of Multiple Reactors Containing Sample Spotting System

The testing device of chip of multiple reactors prepared in this implementation contains sample spotting system (FIG. 5), which consists of the following components: contact tip (26), the fixturn of multiple spotting tips (24) and multi-well plate, wherein: spotting tip includes solid tip (e.g. needle tip, cast-shaped tip etc.) or/and hollowed tip (e.g. capillary tube etc.) or/and water absorbent tip (e.g. fiber stick, paper stick etc.). In this implementation, the tip can be an inlaid tip such as the plastic tip of the pipette and the home-made plastic stem containing a mortise where the fixturn of spotting tips is inlaid. The said home-made plastic stem has a solid tip, which may either have a inlet slop or not.

The said fixturn of spotting tips includes multiple stems and the mobile sheath nearby which can help push out the spotting tip (similar to the stem and sheath on the pipette), wherein each stem should be corresponding to the different pore of the multi-well plate and to the reactor to be spotted.

The said sample spotting system prepared in this implementation is featured by: a). the sample spots are surrounding the center of probe region and the nearby area. b). the spotting tip should be big enough so that the area covered by sample spots on the bottom of reactors should be 1.5-5.0 times of the area of probe region; optionally c). the spotting tip should drop at such a speed that the linear speed of samples subjected to probe region is over 0.1 cm/second.

The sample spotting system prepared in this implementation works in this way (FIG. 5): the N stems (25) on the fixturn (24) will be inlaid to the N spotting tips (26) so that all the bottoms of spotting tips are arranged approximately on the same plane. Then move the fixturn of the multiple spotting tips so that the N tips plunging in N wells on the multi-well plate will have a contact with samples and suck in sample solution as in capillary phenomenon. Then move the fixturn again so that the N tips loaded with samples will contact the N reactors on the chip and spot the samples on the reactors. Finally, move the fixturn away from the chip.

In the spotting system prepared in this implementation, spotting tip can also be linked to the fixturn in other ways, such as by inlaying the spotting tip into the fixture.

The sample spotting system prepared in this implementation can be operated either manually or automatically.

The sample spotting system prepared in this implementation may contain only one spotting tip. Even so, this sample spotting system is essentially different from the probe spotting system based on immobilized probes (e.g. spotting machine) in both objective and technical design. The said sample spotting system is intended to subject the samples to the probe region as designed, while the probe spotting system is intended to subject the probes to the substrate so as to form a probe region. The different objectives thus lead to different technical design. Since the probe spotting system of this invention is intended to form a probe region with probes as densely located as possible, the size of spotting tips should be less than 0.25 mm², usually the smaller the better. However, since the sample spotting system of this invention is intended to form a liquid sample in the reactors containing the whole probe region, the size of the spotting tip should be big enough, such as over 1 mm², preferably over 2 mm².

In the sample spotting system prepared in this implementation, the tip used in spotting can be of round, rectangle or other geometric shapes (FIG. 5b), with the surface area of the tip ranging from 1 mm² to 16 mm². The volume of sample spotted by the tip ranges from 1 μl to 15 μl.

All the devices in this implementation are available by common preparation or from market.

With the said sample spotting, samples are loaded simultaneously to several reactors (e.g. 16-48 reactors), especially the reactors with high density (e.g. multiple reactors with a density over 1 reactor/cm²). In this way, samples can be subjected to multiple reactors with definite volume, contact area and momentum, so that the area and volume of sample can be minimized

Implementation 4: A Testing Device Containing Reactor Cleaning System and Fluidity-Decreasing System

The testing device prepared in this implementation contains the reactor cleaning system prepared in implementation 1 and fluidity-decreasing system prepared in implementation 2. The different systems are connected in the conventional way and controlled by the central controller.

The testing device prepared in this implementation can either include a reaction controlling system or not. It contains reactor cleaning system, fluidity-decreasing system by temperature/moisture controlling and reaction controlling system, which works in this way:

First, open the separate washing chamber (16), put into separate washing chamber the chip with N (N>1) open reactors loaded with sample, and then close the separate washing chamber. Secondly, adjust the temperature/moisture in the washing chamber as required by the temperature/moisture controller. After the reaction is completed within the required time, adapt the temperature/moisture by the temperature/moisture controller to the need of fluidity decreasing (e.g. maintaining the temperature and cut off the moisture supply), then clean the reactors according to the working principle of the testing device with reactor cleaning system and reaction controlling system prepared in the implementation 1.

The testing device prepared in this implementation can either include the marking system in the implementation l or not.

All preparations in this implementation are conventional preparations. The temperature/moisture controllers are selected from the products available on market, while other components and auxiliary parts can be obtained as in the implementation land 2.

Implementation 5: A Testing Device Containing Reactor Cleaning System and Sample Spotting System

The testing device prepared in this implementation contains the reactor cleaning system prepared in implementation 1 and the sample spotting system prepared in implementation 3. The different systems connected with each other in the conventional way, are under control of the central controller.

The testing device prepared in this implementation can either contain a reaction controlling system or not. The testing device prepared in this implementation, which contains reactor cleaning system, sample spotting system and reaction controlling system, works in this way: first, subject the sample by spotting following the working principle of sample spotting system prepared in the implementation 3, then transfer the chip loaded with samples to separate washing chamber by the transporting device. After that, conduct the reaction and reactor cleaning just following the working principle of the testing device containing reactor cleaning system and reaction controlling system prepared in the implementation 1.

The testing device prepared in this implementation can either include the marking system in implementation 1 or not.

All preparations in this implementation are conventional preparations. The temperature/moisture controllers are selected from the products available on market, while other components and auxiliary parts can be obtained as in the implementation 1 and 3.

Implementation 6: A Testing Device Containing Reactor Cleaning System, Fluidity-Decreasing System and Sample Spotting System

The testing device prepared in this implementation contains the reactor cleaning system prepared in implementation 1, the fluidity-decreasing system prepared in implementation 2 and the sample spotting system prepared in implementation 3. The different systems connected with each other in the conventional way, are under control of the central controller.

The testing device prepared in this implementation can either contain a reaction controlling system or not. The testing device prepared in this implementation, which contains reactor cleaning system, sample spotting system and reaction controlling system, works in the following way:

First, subject the sample by spotting following the working principle of sample spotting system prepared in the implementation 3, then conduct the reaction, the fluidity decreasing and reactor cleaning just as with the testing device containing reactor cleaning system, fluidity-decreasing system by temperature/moisture controlling and reaction controlling system prepared in the implementation 4.

The testing device prepared in this implementation can either include the marking system in implementation 1 or not.

All preparations in this implementation are conventional preparations. The temperature/moisture controllers are selected from the products available on market, while other components and auxiliary parts can be obtained as in the implementation 1, 2 and 3.

Implementation 7: A Testing Device with Reactor Cleaning System, Fluidity-Decreasing System and Signal Detecting System

The testing device with signal detecting system, reactor cleaning system prepared in implementation 1 and fluidity-decreasing system in implementation 2, comprises the following systems: a). signal detecting system; b). chip transporting system; c).producing and transporting system of pressured fluid; d). sewage discharging system; e). anti-leaking system; f). reaction controlling system; g). marking system; h). fluidity-decreasing system with fluidity-decreasing additives; i). controlling system.

In this implementation, all the systems except the signal detecting system are similar to or the same as the corresponding sub-systems in implementation 1 or 2. In this implementation, the signal detecting system comprises a fluorescent scanner, connected in string with the reactor cleaning system by chip transporting system.

The testing device prepared in this implementation, which comprises the reactor cleaning system, the fluidity-decreasing system, the signal detecting system, the reaction controlling system and the marking system, works in this way:

The following steps are carried out according to the working principle of the testing device prepared in implementation 4 with reactor cleaning system, fluidity-decreasing system by temperature/moisture controlling and reaction controlling system: 1) conduct the sample/probe reaction; 2) increase the viscosity of the remnant sample; 3) wash the remnant sample; 4) subject marker solution; 5). complete the process of labeling through marking reaction and wash the reaction residual. After that, the dried chip will be transferred by the transporting system to the fluorescent scanner for signal scanning under preset condition.

All preparations in this implementation are conventional preparations. The scanner used here is a fluorescent scanner (SCAN-2s Confocal Laser Biochip Scanner, Chengdu Institute of Optics and Electronics, China Academy of Science); the temperature/moisture controllers are selected from the products available on market, while other components and auxiliary parts can be obtained as in the implementation 1 and2.

Implementation 8: A Testing Device With Reactor Cleaning System, Fluidity-Decreasing System and Other System

The testing device prepared in this implementation, containing signal detecting system, reactor cleaning system prepared in implementation 1 and the fluidity-decreasing system in implementation 2, includes the following systems: a). background signal intensifying structure located in the signal detecting system; b). background signal weakening structure located in the signal detecting system; c). signal detecting system; d). chip transporting system; e). producing and transporting system of pressured fluid; f). sewage discharging system; g). anti-leaking system; h). reaction controlling system; i). marking system; j). fluidity-decreasing system with fluidity-decreasing additives; k). controlling system. All other systems in this implementation are the same as that in implementation 3, except background signal intensifying structure and background signal weakening system.

In another invention of ours (PCT/CN03/01091), a testing method has been provided, which can increase the testing sensitivity by maximizing the chromatic aberration between the background and the target spots in the chip reactors.

In this implementation, the background signal intensifying structures include the paint, wafer or/and lamina with luminescent agent.

In this implementation, the said luminescent agent is selected from fluorescent materials. One of the said background signal intensifying structures is a chip support coated with fluorescent paint available in market. In the fluorescent scanner, this kind of support is used to immobilize the chip and intensify the background signal in the chip reactors while scanning.

In this implementation, the background signal weakening structure is composed of the paint, wafer or/and lamina with light absorbency over 95%, preferably over 97% (or reflecting rate below 5%, preferably below 3%) . The said background signal weakening structure in this implementation is a chip support coated with super black paint (with the light absorbency over 96%). In the fluorescent scanner, this kind of support is used to immobilize the chip and weaken the background signal in the chip reactors while scanning.

The testing device prepared in this implementation, which comprises the reactor cleaning system, the fluidity-decreasing system by temperature/moisture controlling, the signal detecting system, the background signal intensifying structure & weakening structure located in the signal detecting system, the reaction controlling system and the marking system, works in this way:

First immobilize the said chip support in the fluorescent scanner, then the following steps will be carried out as in the testing device prepared in the implementation 4 with reactor cleaning system, fluidity-decreasing system by temperature/moisture controlling, signal scanning, reaction controlling system and marking system: conduct the sample/probe reaction, decrease the fluidity of the remnant sample, wash the remnant sample, subject marker solution, complete the process of labeling through marking reaction and wash the reaction residual. After that the dried chip will be transferred to the scanner for signal scanning.

All preparations in this implementation are conventional preparations. The said chip support is self-made, while other components and auxiliary parts can be obtained as in implementation 1 and 2.

With the same preparation, we can also prepare the chip testing device containing reactor cleaning system, fluidity-decreasing system and sample spotting system.

Implementation 9: A Chip With Multiple Open Reactors Containing a Hydrophobic-Lipophobic Partition Structure

In the following implementations, the glass slides are bought from ESCO SCIENTIFIC in USA, with the glass slide of 75×25×1.0 mm, and the cover glass of 60×24×0.15 mm. The probes are bought from the Research Institute of Hepatopathy, Beijing People's Hospital, which are HIV₁₊₂ antigens syphilis antigen and HCV antigen respectively, with sampling concentration between 1.0-1.5 mg/ml. All the samples are pre-detected with the classic ELISA method. Sample 1 refers to the HCV antibody positive serum, sample 2 refers to the HIV₁₊₂ antibody positive human serum, sample 3 is the syphilis antibody positive human serum and sample 4 is the negative control.

The testing device prepared in this implementation, is a chip of multiple open reactors whose reactor partition structure contains hydrophobic-lipophobic structure. The said partition structure in this implementation is either wholly or partially hydrophobic-lipophobic structure. Here we will only cite the simplest case as an example.
i. preparation of substrate
   1. The preparation of conventional substrate

In this implementation, all the conventional substrates used herein are the self-made amino and epoxy carry glass slide by the well-known amination and epoxylation.

(2) The Preparation of the Nano-Structured Substrate

In this implementation, the nano-structured substrate is prepared in the same way as in some other inventions (PCT/CN2004/000077, PCT/CN2004/000203 and PCT/CN2004/000437). In brief, after the glass slide is soaked for 2 hours in the suspension with 1/10000(w/v) of silicon oxide particles (LUDOX AS—40, Sigma-Aldrich), it is washed, dried and put in the solution with 1/5000(w/v) of polyvinylpyrrolidone for another 2 hours. Finally, after another washing and drying, the substrate will undergo heat treatment (at 60° C. for over 10 hours). Thus the nano-structured substrate has been ready.

The nano-structured substrate prepared in this implementation is evaluated by Scanning Probe Microscope(DFM SPA—300HV) and its analytic software. The density of solidified nano-structure on the substrates is over 10/μm² (whose height is over 3 nm, of which at least one-dimensional size of the convex at the half height is between 1-500 nm, preferably 1-100 nm).

2. The Preparation of the Multi-Well Substrate

The hydrophobic-lipophobic materials used in this implementation are all available in market. The 3 liquid materials used in this implementation are the followings: CityClean by Shenzhen City-Clean Science & Technology Co. Ltd, a highly hydrophobic silicon oxide paint by Zhoushan Minzhi Nanomaterial Company of China, and a hydrophobic-lipophobic paint by China Languang Chemical Institute. The 3 solid materials used in this implementation are 3 forming plates dried as suggested by the supplier after being coated respectively with the 3 liquid hydrophobic-lipophobic materials on one side of the multi-well plate. The depth thickness of the forming plate is less than 0.3 mm.
1. preparation of the multi-well substrate by solidification of the hydrophobic-lipophobic liquid materials

The preparation includes the following steps:

a). paint the hydrophobic-lipophobic liquid material to the partition area on the conventional substrate and the prepared nano-substrate respectively; and

b). solidify the materials to form partition convex according to the instruction of suppliers, or by adding conventional solidifying agent according to the established practice in this field (FIG. 1-(3)).

The hydrophobic-lipophobic convex can be of different shape. In this implementation, the convex is in the shape of belt, 25-115 μm high and 2.0-2.5 mm wide. The surface surrounded by the hydrophobic-lipophobic convex, can also be of different shape. In this implementation, it is a 3 mm×3 mm rectangle. There are altogether 20 wells on the multiple-well substrate, 10 wells in horizon and 2 wells in vertical.

(2). Preparation of the Multi-Well Substrate by Using the Hydrophobic-Lipophobic Solid Material

The preparation includes the following steps:

a). paint the hydrophobic-lipophobic liquid material to the partition area on the preset forming plate;

b). solidify the materials to form hydrophobic-lipophobic coating on the forming plate according to the instruction of suppliers, or by adding conventional solidifying agent according to established practice in this field.

The forming plate can be made of one or more of the following materials: cellulose membrane, paper, plastic and metal etc. In this implementation, the forming plate is a plastic of 75×25×0.45 mm, with 20 rectangular holes (3 mm×3 mm), arranged in 2×10 array, with a distance of 2.0-2.5 mm between two holes. The hydrophobic-lipophobic coating is located at the inner edge of the holes and the separating zone between holes on the surface of the forming plate. Since the lower surface of the forming plate is not covered by hydrophobic-lipophobic coat, it can adhere to the substrate so as to form a multi-well substrate with hydrophobic-lipophobic partitions.

According to the preparation method of this implementation, various multi-well substrates can be prepared, with partition structure at different height (e.g. 0.05-0.5 mm), in different shapes (e.g. belt or other geometric shapes) and of different size; and with reaction wells in different shapes (e.g. rectangular, round, or oval etc.), of different size, numbers and arrangement (e.g. 56 wells with 14 in horizon and 4 in vertical or 16 wells with 8 in horizon and 2 in vertical.

According to the preparation method of this implementation, the substrate can be made of any of the materials, which can immobilize the hydrophobic-lipophobic chips either directly (by the said solidification of the hydrophobic-lipophobic liquid) or indirectly (by immobilization of the hydrophobic-lipophobic solid).

(3). Evaluation of the Multi-Well Substrate with Hydrophobic-Lipophobic Structure

In this implementation, sterile distilled water and purified peanut oil are used respectively for measuring the water contact angle and the oil contact angle of the hydrophobic-lipophobic surface in the partition structure.

The measurement of the contact angle is performed in Chenguang Research Institute of Chemical Industry, with the contact angle analyzer (JC2000A, Shamen Caremeter Scientific Instrument Company). In this implementation, all the water contact angles of the substrates are less than 48°, while the oil contact angles are below 45°. The water contact angles of the hydrophobic-lipophobic surface are all over 100° (e.g. the water contact angle of the silicon oxide paint is over 110°) and the oil contact angles are over 80° (e.g. the oil contact angle of the CityClean is over 100°)

3. Preparation of the Analytic Chip of Multiple Reactors Comprising Hydrophobic-Lipophobic Partition Structure

In fact, to prepare the chip in this implementation, we can either immobilize probes first or prepare the hydrophobic-lipophobic convex on the substrate first. In this implementation, the latter method is adopted.
1. Preparation of conventional chip

With the well-known probe spotting technique (in this implementation, manual spotting is adopted), the three said antigens are immobilized respectively to the central area (over 0.5 mm away from the hydrophobic-lipophobic structure) of the said multi-well substrate, to form a 3×3 probe array, with 3 spots for each antigen. Once the immobilizing reaction is over, the cow serum albumin is used to block the chip for future use.

(2) Preparation of the Chip with Active Nano-Structure

one preparation method is to immobilize the said antigens respectively to the central area (over 0.5 mm away from the hydrophobic-lipophobic convex body) of the multi-wells in the said conventional nano-structured substrate to form a 3×3 probe array, with 3 spots for each antigen. Once the immobilizing reaction is over, the cow serum albumin is used to block the chip for future use.

Another preparation method refers to some other inventions of ours, such as PCT/CN2004/000077, PCT/CN2004/000203 and PCT/CN2004/000437. In brief, mix the suspension containing nano-particles of silicon oxide (LUDOX AS—40, Sigma-Aldrich) respectively with the said antigens so that after reaction, the said antigens are immobilized on the nano-particles, then with established probe spotting, the three antigens are immobilized to the central area (over 0.5 mm away from the hydrophobic-lipophobic structure) of the multi-wells in the said conventional nano-structured substrate to form a 3×3 probe array, with 3 spots for each antigen. Once the immobilizing reaction between antigen and silicon oxide particles is over, the cow serum albumin is used to block the chip for future use.

4. Evaluation of the Multi-Reactor Analytic Chip with Hydrophobic-Lipophobic Partitions

The evaluation method comprises the following steps:

a). taking 10 of each said chip in which the reactors are numbered;

b). subjecting to the first row reactors of odd number the positive serum, made by mixing together the same amount of sample 1, 2 and 3, and to the reactor of even number the negative control serum, the second row is reverse, subjecting to the even number the positive serum, and the odd number the negative control serum, the third row is as the same of first row, and the forth row is as the same of second row.

The marker used herein is the self-made mouse mAb of anti human IgG labeled by rhodamine. In the experiment, 10 μl of samples and 10 μl of markers will be subjected. After the reaction, it is not necessary to suck the non-mobilized materials before washing as does in ELISA washing. Instead, the following washing method is adopted:

a). absorb the non-mobilized materials by making the reaction well have a contact with water absorbent paper, then wash the well by pressing on the washing sprayer

b). rotate the glass slide so that an angle of 45° is formed between it and the horizontal, then cleaning it by spraying washing fluid from above with the cleaning system prepared in the implementation 1;

c). rotate the glass slide so that an angle of 180° is formed between it and the plane, then cleaning it by spraying washing fluid zenithward with the cleaning system prepared in the implementation 1.

Then subject marker, conduct reaction, washing and drying as in the established practice. Scanning is conducted after drying. The scanner used in this implementation is a laser scanner GMS 418 made by Affymetrix, whose wave-length of excitation light is 532 nm and that of emission light is 570 nm. The detected signals are progressed by JAGUAR II. The rate of cross-contamination is defined as the number of wells whose results are inconsistent with the samples added over the total number of wells under investigation. The results are shown in table 5.

TABLE 5
Evaluation Results of the analytic chips with different partition structures

| | Hydrophobic- | | Depth of | | | | Rate of |
|---|---|---|---|---|---|---|---|
| | lipophobic | | convex | | | | cross- |
| chip | materials | substrate | body | | WCD* | OCD** | contamination |
| 1 | CityClean | Epoxy glass | 75-105 | μm | >60° | >50° | 0 |
| | | slide | | | | | |
| 2 | CityClean | Epoxy glass | 20-115 | μm | >60° | >50° | 0 |
| | | slide | | | | | |
| 3 | CityClean | Epoxy glass | 20-89 | μm | >60° | >50° | 0 |
| | | slide | | | | | |
| 4 | CityClean | Epoxy glass | 15-106 | μm | >60° | >50° | 0 |
| | | slide | | | | | |
| 5 | Silicon oxide | Epoxy glass | 75-105 | μm | >70° | >30° | 0 |
| | paint | slide | | | | | |
| 6 | Silicon oxide | Epoxy glass | 65-89 | μm | >70° | >30° | 0 |
| | paint | slide | | | | | |
| 7 | Hydrophobic- | Epoxy glass | 120 | μm | >50° | >30° | 0 |
| | lipophobic | slide | | | | | |
| | paint | | | | | | |
| 8 | Hydrophobic- | Epoxy glass | 400 | μm | >50° | >30° | 0 |
| | lipophobic | slide | | | | | |
| | forming plate | | | | | | |
| 9 | Hydrophobic- | Black glass | 400 | μm | >50° | >30° | 0 |
| | lipophobic | slide | | | | | |
| | forming plate | | | | | | |

WCD* water contact angle difference between hydrophobic-lipophobic material and substrate
OCD** oil contact angle difference between hydrophobic-lipophobic material and substrate

In this implementation, the chip of multiple reactors is highly integrated in structure. For instance, a 25×75 mm (width×length) chip may consist of several reactors (e.g. 16-48 reactors or even more). It is also highly integrated in application. For instance, it can be applied to the highly integrated testing in this invention. Besides, the reactors on the chip are of a high density (e.g. there are over 1 or even more reactors on every 1 cm² of substrate), which can greatly reduce the cost of testing.

The kit containing the invented chip can achieve the same result as the said chip of multiple reactors in this invention.

Implementation 10: A Chip with Multiple Reversibly Closed Reactors

The chip with multiple reversibly closed reactors, prepared in this implementation, is a chip with multiple reversibly-closed spacious-chamber reactors.

In this implementation, the chip with multiple reversibly-closed wet chamber is a chip prepared according to another invention of ours PCT/2004/001128 while introduced with the hydrophobic-lipophobic partition structure of this invention. In brief, it is a combination of top component and bottom component that form a chip of multiple exposed reactors.

In this implementation, the bottom component comprises bottom substrate with inlet area and outlet area, probes, hydrophobic-lipophobic partition structure and the chamber wall made of hydrophobic-lipophobic structure. To prepare the bottom components mentioned above, we would paint the said hydrophobic-lipophobic liquid materials evenly to all the areas on the substrate other than the 8 bottom areas (12 mm×4 mm for each), reserved to immobilize probes. When the paint is dried after a whole night, a partition structure of elastic material will come into being. After that, spot the said antigen solution in the reserved area with GM 417 ARRAYER by GENETIC MICROSYSTEMS, to form a probe array of 3×3, with 3 spots for each ligand. The reaction will be incubated at room temperature for 3 hours, and then the chip is washed and dried for future use after enclosing with calf serum.

In this implementation, the top component includes top (as opposed to bottom), inlet/outlet of spacious chamber, inlet structure of reactors, outlet structure of reactors, positioning structure and partition structure, wherein: the inlet structure refers to the inlet pipe, while the outlet structure refers to outlet pipe which can be easily accessed to pipelines in other systems (fluid transporting mechanics). The said top component is a stainless steel plate of 100 mm×40 mm×2 mm (length×width×depth), made first through mechanical processing, then coated with elastic solution (e.g. silastic solution made by Chenguang Research Institute of Chemical Industry) at the depth of less than 0.18 mm. The said elastic solution is painted on the corresponding partition structures of the bottom and the top component. Each pair of inlet and outlet is corresponding to the inlet and outlet area in each reactor of the bottom component. In a word, the partition structures of the bottom component and that of the top component are corresponding to each other. The top component is available for repetitive use.

A close chip with multiple wet chambers is a combination of the said top component with said bottom component by mechanical pressure of fixturn, which can be used for sample loading and sample-probe reaction. Said bottom component will become a chip with multiple exposed reactors, when remnant samples are sucked out of each chamber after reaction, and top component is separated from bottom component as the mechanical pressure of fixturn is removed.

In the wet chamber prepared in this invention, each of the multiple close wet chambers has a bottom of 12 mm×4 mm, a wall about 0.25 mm high, wherein it takes the anionic water less than 1 second or even less than 0.5 second to fill in said spacious chamber horizontally through moisture phenomenon.

Implementation 11: A Chip with Multiple Open Reactors

In this implementation, a chip with multiple open reactors will be prepared. Here illustrated is the simplest case.

In effect, the chip in this invention can be made by immobilizing probes on the substrate first and then immobilizing the partition structure or by immobilizing partition structure on the substrate first and then probes. The two procedures are of the same working principle but opposite sequence. In this implementation, the latter one will be adopted.

Preparation of Substrate with Multiple Wells

In this implementation, the substrate in use refers to the conventional substrate or the nano-structured substrate prepared in implementation 9. Though the partition structure in this invention can be either partially or totally composed of hydrophobic, superhydrophobic or hydrophobic-lipophobic materials, we will only exemplify the simplest case in this implementation.

(1). Preparation of Multi-Well Substrate with Hydrophobic Partition Structure

The hydrophobic material used in this implementation is the black polyethylene plastic or black polyvinyl chloride plastic available in market. The hydrophobic material is molded through heat treatment into forming plate of 75.0×25.0×0.5 mm (length×width×height) with 16 holes arranged in two rows eight columns whose diameter is 5 mm. The static water contact angles of the two forming plates are 78 and 73 respectively. The substrate with 16 wells is formed when substrate and forming plate are adhered together.

(2). Preparation of Multi-Well Substrate with Superhydrophobic Partition Structure

The superhydrophobic liquid materials used in this implementation are polyacrylate paint (by Chenguang Research Institute of Chemical Industry, with static water contact angle of 85°), waterproof paint of organic silicon (by Chenguang Research Institute of Chemical Industry, with static water contact angle of 116°) and superhydrophobic silicon oxide paint (by Zhoushan Minzhi Nano-material Company, with static water contact angle of 151°). The superhydrophobic solid materials used in this implementation are politef PTFE adhesive tap (by Chengdu Chenguang Chemical Research Institute, with static water contact angle of 117°) and nano-textile (by Zhoushan Minzhi Nano-material Company, with static water contact angle of 155°).

In this implementation, the preparative method of multi-well substrate using the materials of solidifying the superhydrophobic liquid:

paint said superhydrophobic materials to the partition area on said conventional substrate and said nano-structurered substrate respectively

solidify the materials to form the superhydrophobic coat by adding proper amount of solidifying agents according to the instruction of suppliers or established practice in this field.

The superhydrophobic coat can be of different geometric shapes. In this implementation, it is of a belt, 85-115 μm high and 4.5 mm wide. The reaction wells surrounded by the superhydrophobic coat can also be of different shape. In this implementation, it is a 4.5 mm×4.5 mm rectangle. There are altogether 16 wells on the prepared substrate, with 8 wells in horizon and 2 wells in vertical.

In this implementation, the multi-well substrate can also be made of the superhydrophobic solid materials, that is, to adhere the molded solid materials to the substrate. The multi-well substrates obtained in this way are only different in its height (from 300 μm to 500 μm).

(3). Preparation of Multi-Well Substrate with Hydrophobic-Lipophobic Partition Structure

The multi-well substrate with hydrophobic-lipophobic partition structure in this implementation is prepared as that in implementation 5, wherein: in the multi-well substrate prepared by immobilizing hydrophobic-lipophobic solid materials, the partition structure is 300-600 μm high, 4.5 mm wide; while in the multi-well substrate prepared by solidifying hydrophobic-lipophobic liquid materials, the coat is 100-300 μm high, 4.5 mm wide. The prepared substrate in this implementation has 16 wells altogether, with 8 in horizon and 2 in vertical, each well in shape of a 4.5 mm×4.5 mm rectangle.

2. Preparation of Analytic Chip with Multiple Open Reactors

In this implementation, the conventional chip or the chip with active nanostructure is prepared as the conventional chip or the chip with active nanostructure in implementation 9, except that the 3×3 probe array is spotted by DY—2003 biochip spotting instrument made by Beijing Electro-industrial Research Institute, which covers an area of less than 1 mm². If more spots are needed, other high-density spotting instrument can be used such as the one with spotting density over 50 probe-spots/mm². The chip prepared herein refers to the chip with multiple open reactors.

3. Preparation of the Chip with Multiple Protective Reactors

The chip with multiple protective reactors can be prepared with the chip of multiple reactors prepared in the foregoing implementations, which can refer to another invention of ours (PCT/CN2004/000169). In brief, that is to adhere the polyethylene lemma (which is cut preliminarily so that the parts cover the reactors of the lemma can easily be torn open) to central line of the partition area on the said chip. The enclosing component in this implementation includes the mechanically processed plastic film and aluminium-plastic film with detachable areas, and the plastic film either with or without detachable area.

4. Evaluation of Analytic Chip with Multiple Open Reactors
1. evaluation of partition effect

The evaluation is conducted by opening the protective system in the said chip with multiple protective reactors, or directly by using the analytic chip with multiple open reactors. To be more exact, researchers should:

First place the analytic chip with multiple open reactors onto a plane support, then transfer 3-5 μl anionic distilled water with a pipette to the probe region in each reaction well so as to form a semi-spherical water drop whose bottom area is 2 times over that of the probe region, with height over 150 μm. Second, with the angle adjustor prepared in implementation 1, which is connected with the support, the chip in this implementation is gradually turned around clockwise with the longest midline as axis. Then examine it to see if any anionic distilled water will spill out of the reaction well. No anionic distilled water is observed to spill out the well in the analytic chip prepared in this implementation, which suggests that the height and surface hydrophobicity of partition structure and the area of probe array are properly arranged herein.

(2). Assay of Cross-Contamination Rate

In this implementation, the cross-contamination rate is detected in the same way as that in implementation 9 and bears the same result.

In this implementation, the chip of multiple reactors is highly integrated in structure. For instance, a 25×75 mm (width×length) chip may consist of several reactors (e.g. 16-48 reactors or even more). It is also highly integrated in application. For instance, it can be applied to the highly integrated testing in this invention. Besides, the reactors on the chip are of a high density (e.g. there are over 1 or even more reactors on every 1 cm² of substrate), which can greatly reduce the cost of testing.

The kit containing the invented chip can achieve the same result as the invented chip of multiple reactors.

Implementation 12: A Testing Method with Chip of Multiple Reactors (1)

In this implementation, the devices used are the testing devices prepared in the implementation 9 and 10, and the chips used herein are the chips with multiple reactors prepared in the above implementations.
i. using chip of multiple reactors

In this case, the said chip of multiple reactors is the chip with multiple open reactors prepared in implementation 9. In application, the protective structure, if any, should be removed, or the chip without protective structure can be used directly.
1. using the testing device with cleaning system only

The testing device in this implementation is the same testing device of chip of multiple reactors prepared in implementation 1.

First, subject the said samples that have already been diluted 20 times with PBS buffer solution to the multiple wells on the ELISA micro-well plate corresponding to the reaction wells on the chip; then the sample is loaded either by the conventional sampling or by the invented spotting. If spotting is chosen, the sample spotting system in implementation 3 should be employed. In the process of sampling, first, multiple contact tips in proper shape and size are immobilized to the stems corresponding to the reaction wells on the fixturn, then soak the spotting tips into the corresponding wells on the micro-well plate. Then 1-5 μl sample will be subjected to each reactor in the said analytic chip by stamping with the tip evenly covered with sample. As a result, a spotting area of 2-3 mm² will form on the bottom of reactor. The spotting tip will go down at the speed of about 10 cm/second.

After that, the chip is kept at 37° C. for 1 hour. After the reaction, open the separate washing chambers in the testing device and put the said chip upside down onto the chip support therein, then close the separate washing chamber. Turn on pressure pump to press the PBS solution stored at the room temperature in the washing solution bottle to No. 4 nozzle at an optimal position, which will produce 16 independent washing fluids through 16 outlets onto 16 reactors.

The spurting parameters are listed as below:

Every outlet on the nozzle is targeting at a probe array in the reaction well.

The clockwise angle between the individual fluid at the outlet and the substrate of said chip immobilized with probes should be 90±5 degree, even if the fluid is spurting upward.

The distance between the said outlet and the substrate immobilized with probes is adjusted to 2.0±0.3 cm (the depth thickness of said chip in this implementation is 1.0±0.1 mm).

the working pressure of the pump is adjusted to 2.5±0.5 kg/cm².

e. Spurting will last for 10 seconds

These washing fluids will reach the immobilized probes in the exposed reactor and then splash, which will take out the remnant samples and clean the exposed reactor. All the splashed fluid will be discharged into the pool for liquid waste through drainage pipe. Finally, open the separate washing chamber and take out the chip.

If fluid-cleaning system by pressure/ultrasound is used, the ultrasound generator (e.g. that with the power of 30 W) is connected between pressure pump and nozzle.

After blowing the chip dry, add to the reactor the marker solution (goat antibody against human IgG+IgM+IgA labeled with rhodamine) and maintain the reaction at 37° C. for 1 hour. When the reaction is over, put the said chip upside down on the chip support in the separate washing chamber and then repeat the above washing procedure.

The dried chip is then scanned and analyzed by the confocal laser scanner (GMS 418, by Affymetrix), with the following scanning parameters: excitation wavelength and emission wavelength are 532 nm and 570 nm respectively. The intensity/gain of the laser are 60/69 respectively. The detected signals are processed by software JAGUAR II, with average value as the final result. The positive or negative result of each reactor turns out to be consistent with the sample added. In this implementation, 20 chips have undergone the said chip testing for 20 times successively with the same result that no cross-contamination is observed. However, the reactors sampled by spotting bear more positive results than by the conventional sampling, which indicates a higher sensitivity.

(2) Using Testing Devices With Cleaning System and Reaction Controlling System

The testing device used in this implementation refers to the testing device of chip of multiple reactors prepared in implementation 1.

First, subject to each open reactor of the said analytic chip 10 μl said sample that has already been diluted 20 times with PBS buffer solution; then put the said chip plainly onto the chip support in the separate washing chamber with the right side up and close the chamber. Set the temperature in the separate washing chamber to 37° C. and moisture level there to 85%, then maintain the reaction for 1 hour. After the reaction, turn the chip upside down by adjusting the angle of the chip support, then turn on pressure pump to press the PBS solution stored in the washing solution bottle to No. 4 nozzle, which will produce 16 independent washing fluids through 16 outlets onto 16 reactors. The spurting parameter herein is the same as that in the said testing device with cleaning system only; and the results are also the same.

(3) Using the Testing Device with Cleaning System, Fluidity-Decreasing System by Controlling Temperature/Moisture, Reaction Controlling System and Marking System

The device in this implementation refers to the same testing device of chip of multiple reactors prepared in implementation 4.

First, subject to each open reactor of the said analytic chip 10 μl said sample that has already been diluted 20 times with PBS buffer solution; then put the said chip plainly onto the chip support in the separate washing chamber with the right side up and close the chamber.

Set the temperature in the separate washing chamber to 37° C. and moisture level there to 85%. After maintaining the reaction for 1 hour, decrease the moisture level to 60% for 3-5 minutes, then turn the chip upside down by adjusting the angle of the chip support, and turn on pressure pump to press the PBS solution stored in the washing solution bottle to No. 4 nozzle, which will produce 16 independent washing fluids through 16 outlets onto 16 reactors. The spurting parameter herein is the same as that in the said testing device with cleaning system only. After being cleared of reaction residuals and being dried, the chip will again be turned upside down by adjusting the angle of the chip support. Subject to each reactor 10 μl marker solution (goat antibody against human IgG+IgM+IgA labeled with rhodamine) and keep the reaction at 37° C. for 1 hour. After the reaction, turn the chip upside down by adjusting the angle of the chip support, and turn on pressure pump to repeat the above washing procedure.

Other operations herein are the same as that in the said testing device with cleaning system only; and bear the same results.

4) Using the Testing Device With Cleaning System, Fluidity-Decreasing System With Additive, Reaction Controlling System and Marking System

The device in this implementation refers to the same testing device of chip of multiple reactors prepared in implementation 4.

First, subject to each open reactor of the said analytic chip 10 μl said sample that has already been diluted 20 times with PBS buffer solution; then put the said chip plainly onto the chip support in the separate washing chamber with the right side up and close the chamber.

Set the temperature in the separate washing chamber to 37° C. and moisture level there to 85%. After maintaining the reaction for 1 hour, add to each reactor lmg glucose powder. Then turn the chip upside down by adjusting the angle of the chip support, and turn on pressure pump to press the PBS solution stored in the washing solution bottle to No. 4 nozzle, which will produce 16 independent washing fluids through 16 outlets onto 16 reactors. The spurting parameter herein is the same as that in the said testing device with cleaning system only.

After being cleared of reaction residuals and being dried, the chip will again be turned upside down by adjusting the angle of the chip support. Subject to each reactor 10 μl marker solution (goat antibody against human IgG+IgM+IgA labeled with rhodamine) and keep the reaction at 37° C. for 1 hour. After the reaction, turn the chip upside down by adjusting the angle of the chip support, and turn on pressure pump to repeat the above washing procedure.

Other operations are the same as that in the said testing device with cleaning system only and bear the same results.

(5) Using the Testing Device with Cleaning System, Fluidity-Decreasing System With Additive, Signal Detecting System, Reaction Controlling System and Marking System

The device used in this implementation is the same testing device with chip of multiple reactors prepared in implementation 7.

As in the case of the said testing device with cleaning system, fluidity-decreasing system with additive, reaction controlling system and marking system, we will start sample/probe reaction first, then add additive to decrease the fluidity. After washing reaction residual, marker solution is added to trigger the marking reaction. Then wash the remnant marker and send the dried chip through transporting system to the fluorescent scanner for signal scanning as presupposed. The scanner used in this implementation is a confocal laser scanner SCAN-2 made by Chengdu Institute of Optics and Electronics, China Academy of Science, whose excitation wave-length is 532 nm and that of emission is 570 nm, with laser intensity and gain at 60/69. The scanning result is the same as in above cases.

(6). Using the Testing Device with Cleaning System, Signal Detecting System, Background Signal Intensifying Structure and Weakening Structure in the Signal Detecting System, Reaction Controlling System and Marking System

Device in this implementation is the same as the testing device with chip of multiple reactors prepared in implementation 8.

First, immobilize the background plate coated with fluorescent paint in the fluorescent scanner. Then as in the case of the testing device with chip of multiple reactors containing cleaning system, fluidity-decreasing system with additive, reaction controlling system and marking system, we will start the sample/probe reaction, wash the reaction residual, then add marker solution to trigger the marking reaction. After washing the remnant markers, send the dried chip through transporting system to the fluorescent scanner for scanning as presupposed. The scanner used in this implementation is a confocal laser scanner SCAN-2 made by Chengdu Institute of Optics and Electronics, China Academy of Science, with the scanning parameters as below: excitation wave-length is 532 nm and that of emission is 570 nm, with laser intensity and gain at 60/69. The scanning result is the same as in above cases.

2. Using the Chip With Multiple Reversibly Closed Reactors

The chip of multiple reactors used in this implementation is the chip with multiple reversibly closed reactors prepared in implementation 10. With the help of mechanic pressure of fixture, the said top components and bottom components of the chip will be conjugated to form a closed chip with multiple wet chambers.
1. preparation of the multiple exposed reactors containing remnant samples

Heat the said sample diluted 20 times with PBS buffer solution to 37° C., then press the sample with the micropump to each reactor at the speed of 0.05 ml/hour. After about 60 minutes of sampling, turn off the pump then blow the remnant samples dry. When the mechanic pressure of fixture is removed, the top components and the bottom component become separate, thus form a chip with multiple exposed reactors.

(2) Simultaneously Providing at Least One Independent Fluid to Each Said Reactor Which Will Automatically Remove the Said Remnant Samples

Put the said chip the right side down onto the chip support therein, then close the separate washing chamber. Turn on pressure pump to press the PBS solution stored at the room temperature in the washing solution bottle to No. 4 nozzle at an optimal position, which will produce 16 independent washing fluids through 16 outlets onto 16 reactors.

The spurting parameters are listed as below:

Every outlet on the nozzle is targeting at a probe array in the reaction well.

The clockwise angle between the individual fluid at the outlet and the substrate of said chip immobilized with probes should be 90±5 degree, even if the fluid is spurting upward.

The distance between the said outlet and the said probe region is adjusted to 2-3 cm

The working pressure of the pump is adjusted to 2.5±0.5 kg/cm².

e. The Spurting Will Last for 10 Seconds

The washing fluids will splash away after reaching the target probe immobilized on the exposed reactor, thereby taking away the remnant samples from the exposed reactor and washing the reactor. All the splashed liquid will then be collected and transferred to the pool for liquid waste through the drainage pipe. After that, the separate washing chamber will be opened and the chip is taken out.

If fluid-cleaning system by pressure/ultrasound is used, the ultrasound generator with the power of 30 W is connected between pressure pump and nozzle.

After blowing the chip dry, put the said chip the right side up, add to the reactor the marker solution (goat antibody against human IgG+IgM+IgA labeled with rhodamine) and maintain the reaction at 37° C. for 1 hour. When the reaction is over, put the said chip the right side down on the chip support in the separate washing chamber and then repeat the above washing procedure.

The dried chip is then scanned and analyzed by the confocal laser scanner (GMS 418, by Affymetrix). Scanning parameters are: excitation wavelength and emission wavelength are 532 nm and 570 nm respectively. The intensity and gain of the laser are 60/69 respectively. The detected signals are processed by software JAGUAR II, with average value as the final result. The positive or negative result of each reactor turns out to be consistent with the sample added. In this implementation, 20 chips have undergone the said chip testing for 20 times successively with the same result that no cross-contamination is observed.

Similarly, other testing devices prepared in implementation 1-8 can also be used for washing the remnant sample, adding marker solution, starting marking reaction, washing remnant marker, fluorescent scanning and signal analyzing etc. for the chip with multiple exposed reactors.

In this implementation, fluidity decreasing process can be simultaneously conducted on several reactors (e.g. 16-48 reactors), especially the reactors with high density (e.g. with a density over 1 reactor/cm²). It usually takes a very short time (e.g. 1-100 seconds) and can greatly reduce the fluidity of remnant sample.

The one-way cleaning in this implementation can be conducted simultaneously on several reactors (e.g. 16-48 reactors), especially the reactors with high density (e.g. with a density of over 1 reactor/cm²). It usually takes a very short time (e.g. 1-10 seconds) and can greatly enhance the washing effect by increasing the linear speed of the washing fluid (e.g. when the pump pressure is over 5.0 kg/cm², no target signal is lost. Besides, the chip presents a clean background, with the occurrence of marks on the background decreasing by more than 2 times). In this way, the cross-contamination is controllable. In addition, its high repeatability enables the automation.

Implementation 13: A Testing Method With Chip of Multiple Reactors (2)

The testing devices used in this implementation are the same devices in implementation 1-8, while the chip in use is the chip of multiple reactors prepared in implementation 11.

In this implementation, the sample is loaded by spotting: With the sample spotting system prepared in implementation 3, the optimal amount of said samples already diluted 20 times with PBS buffer solution is subjected to each reactor on the said analytic chip with multiple open reactors. The optimal amount of sampling in this implementation is decided in this way: when the sample is subjected, the liquid materials thus formed will cover an area at the bottom of the reactor, which under reaction condition should be 2 times larger than probe region, but smaller than 4 times of the probe region, with maximum height over 150 μm but less than 300 μm. As proved by the experiment, the amount of sampling in the chip of multiple reactors prepared in implementation 11 is 2.5-3.5 μl.

In this implementation, the spotting tip should target at the center of probe region as well as its adjacent area and should go down at such a speed that the linear speed of sample to the probe region is over 5 cm/second.

In addition, other methods and devices adopted in this implementation are the same as that in implementation 12, which uses chip with multiple open reactors. The result is also the same.

This invention provides a testing method of chip of multiple reactors, by which the amount of sampling can be optimized. It has many advantages besides the advantage of the testing method in implementation 12; for instance, it consumes fewer samples and can lower the risk of cross-contamination. When the chips with multiple open reactors prepared in implementation 9 and 11 are tested respectively by adopting the method in implementation 12 and 13, the former has higher contamination rate than the latter within 100 tests.

Besides, compared with the conventional sampling by pipette, the sample spotting method in this invention turns out to be more sensitive on average.

With the said spotting in this implementation, samples are loaded simultaneously to several reactors (e.g. 16-48 reactors), especially the reactors with high density (e.g. multiple reactors with a density over 1 reactor/cm²). In this way, samples can be subjected to multiple reactors with definite volume, contact area and momentum, so that the average sensitivity can be increased and the area and the volume of sample can be minimized

In addition, when the said sample spotting, fluidity-decreasing and one-way cleaning are combined in any way (spotting with fluidity-decreasing procedure, spotting with one-way cleaning, fluidity-decreasing procedure with one-way cleaning or spotting with fluidity-decreasing procedure and one-way cleaning etc.), they can achieve an even better result (e.g. an even higher efficiency or/and higher safety) without sacrificing their own integration, efficiency and safety.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A testing device of analytic chip of multiple reactors, comprising:
a). one-way-cleaning system for one-way-cleaning of remnant sample in said reactor, wherein said one-way cleaning comprises subjecting Q individual fluids to N reactors respectively, by which said remnant samples are washed away from said reactors, wherein Q≧N≧2; or/and
b). fluidity-decreasing system for fluidity-decreasing of remnant sample; or/and
c). sample spotting system for sample subjecting by spotting.

2. The device of claim 1, wherein said Q fluids are subjected respectively to said N reactors at almost the same time.

3. The device of claim 1, wherein said one-way-cleaning system comprises nozzle with Q outlets by which said Q fluids are produced.

4. The device of claim 3, wherein:
a). linear speed of said fluids arriving on said reactors is 1-1000 cm/second; or/and
b). diameter of said outlet is 0.1-1.0 mm; or/and
c). density of said outlets on said nozzle is over 1 unit/cm²; or/and
d). clockwise angle between said individual fluid and probe region of said reactor is 1 to 179 degrees or 135-179 degrees; or/and
e). distance between any of said outlets and its nearest probe region of said reactor is 0.1-10.0 cm.

5. The device of claim 4, wherein said clockwise angle is 90±10 degrees.

6. The device of claim 1, wherein said fluidity-decreasing system consists of at least one of the following systems:
a). temperature controlling system or/and moisture controlling system used to increase viscosity of said remnant sample;
b). loading system for subjecting fluidity-decreasing additive into said remnant sample; and
c). water-absorption system, comprising water absorbent, for decreasing volume of said remnant sample.

7. The device of claim 6, wherein said fluidity-decreasing additive includes chemically/biologically stable additive, including one or more of following groups: carbohydrate, polymer powder, chromatography gel, particle with multiple pores.

8. The device of claim 1, wherein said sample spotting system includes one or more contact-spotting tips for subjecting sample to said reactor by the tip contacting.

9. The device of claim 1, wherein:
a). linear speed of said sample spotted to said reactor is over 0.1 cm/sec; and/or
b). sample spot formed by said contact spotting covers an area on said reactor, which is 1.5 to 5.0 times as large as that of probe region of said reactor.

10. The device of claim 1, also comprising optical-signal detecting system, wherein:
a). said detecting system contains background-signal intensifying system which comprises light radiating or/and reflecting structure in background area of detected chip; or/and
b). said detecting system contains background-signal weakening system which comprises light absorbing structure in background area of the detected chip, whose light absorbency is over 95%.

11. The device of claim 1, also comprising analytic chip of multiple reactors, composed at least of substrate, probe spot immobilized thereon, and reactor partition structure linked thereon, wherein at least part of said partition structure presents a height less than 1 mm.

12. The device of claim 11, wherein said partition structure comprises hydrophobic structure and/or hydrophobic-lipophobic structure, wherein said partition structure comprises hydrophobic-lipophobic structure whose surface is more hydrophobic and lipophobic than that of said substrate.

13. The device of claim 12, wherein:
a). said surface of hydrophobic-lipophobic structure presents a water contact angle 40 degrees more than that of said substrate; and
b). said surface of hydrophobic-lipophobic structure presents an oil contact angle 10 degrees more than that of said substrate.

14. The device of claim 12, wherein said hydrophobic-lipophobic structure contains hydrophobic-lipophobic material.

15. The device of claim 14, wherein said hydrophobic-lipophobic material includes hydrophobic-lipophobic organic material or/and hydrophobic-lipophobic nano-material.

16. The device of claim 11, wherein said reactor contains non-probe region, and probe region that presents a minimized area; and at least part of said partition structure presents a surface that is more hydrophobic than that of said substrate.

17. The device of claim 16, wherein:
a). said minimized area is less than 4.0 mm², and is smaller than the area of said non-probe region; or/and
b). the height ranges of the lowest part of said partition structure from 0.01 mm to 0.80 mm; or/and
c). said surface of partition structure presents a water contact angle between 20 degrees more than that of said substrate surface.

18. The device of claim 17, wherein:
a). said minimized area is less than 1 mm²; and/or
b). said probe region presents a density of more than 9 probe spots/mm².

19. The device of claim 17, wherein said area of probe region, said height of partition structure and said water contact angle are determined in this way that when said chip is rotating 360°, no water sample subjected to said probe region will spill out of any of said reactors.

20. The device of claim 2, wherein said fluidity-decreasing system consists of at least one of the following systems:
a). temperature controlling system or/and moisture controlling system used to increase viscosity of said remnant sample;
b). loading system for subjecting fluidity-decreasing additive into said remnant sample; and
c). water-absorption system, comprising water absorbent, for decreasing volume of said remnant sample.

21. The device of claim 2, wherein said sample spotting system includes one or more contact-spotting tips for subjecting sample to said reactor by the tip contacting.

22. The device of claim 8, wherein:
a). linear speed of said sample spotted to said reactor is over 0.1 cm/sec ; and/or
b). sample spot formed by said contact spotting covers an area on said reactor, which is 1.5 to 5.0 times as large as that of probe region of said reactor.

23. The device of claim 2, also comprising optical-signal detecting system, wherein:
a). said detecting system contains background-signal intensifying system which comprises light radiating or/and reflecting structure in background area of detected chip; or/and
b). said detecting system contains background-signal weakening system which comprises light absorbing structure in background area of the detected chip, whose light absorbency is over 95%.

24. The device of claim 2, also comprising analytic chip of multiple reactors, composed at least of substrate, probe spot immobilized thereon, and reactor partition structure linked thereon, wherein at least part of said partition structure presents a height less than 1 mm.
